(19) United States
(12) Patent Application Publication
(10) Pub. No.: US 2009/0226527 A1
(43) Pub. Date: Sep. 10, 2009

(54) Particulate-stabilized injectable pharmacutical compositions of posaconazole

(76) Inventors: Leonore Witchey-Lakshmanan, Piscataway, NJ (US); Sydney Ugwu, Gurnee, IL (US); Varda E. Sandweiss, New York, NY (US); Catherine Hardalo, Morristown, NJ (US); Roberta S. Hare, Gillette, NJ (US); Gopal Krishna, North Brunswick, NJ (US); Zaiqi Wang, Edison, NJ (US); Marco Taglietti, Watchung, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 12/380,790

(22) Filed: Mar. 3, 2009

Related U.S. Application Data
(63) Continuation of application No. 11/300,677, filed on Dec. 14, 2005.
Application No. 11/300,677 is a continuation-in-part of application No. 11/140,294, filed on May 27, 2005.
(60) Provisional application No. 60/575,126, filed on May 28, 2004.

(51) Int. Cl. A61K 9/14; A61P 31/10

(52) U.S. Cl. 424/489

(57) ABSTRACT

The present invention provides formulations useful for treating infections, in particular, formulations that include the active pharmaceutical ingredient Posaconazole in an injectable suspension of particles that is stable when subjected to terminal sterilization. Preferred median particle sizes of between 1.5 and 3.0 microns are found to result in superior pharmacokinetic characteristics-displayed in FIG. 7.

This application is a continuation-in-part of U.S. patent application Ser. No. 11/140,294 filed May 27, 2005, which claims priority benefits of application No. 60/575,126 filed May 28, 2004, the entire disclosures of which are incorporated herein by reference.

### FIELD OF THE INVENTION

The present invention relates to formulations useful for treating infections. Specifically, these formulations include the active pharmaceutical ingredient Posaconazole in an injectable suspension that is stable when subjected to terminal steam sterilization, and throughout the shelf life of the product. The Posaxonazole particle size distribution of the suspension results in superior pharmacokinetic characteristics.

### BACKGROUND OF THE INVENTION

Posaconazole, an anti-fungal agent, represented by the following chemical structural formula

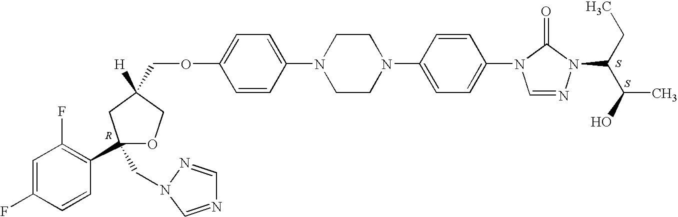

is being developed as an oral suspension (40 mg/ml) under the trademark NOXAFIL® by Schering Corporation, Kenilworth, N.J. See, for example, U.S. Pat. Nos. 5,703,079, 5,661,151, WO 02/80678 published Oct. 17, 2002, and EP 1 372 394 published Jan. 2, 2004. In addition, other formulations of Posaconazole have been disclosed. A solid (capsule/tablet) of Posaconazole is disclosed in U.S. Pat. Nos. 5,972,381 and 5,834,472. Lastly, a topical form of Posaconazole, e.g., a lotion, cream, ointment, or “lacquer nail polish” is contemplated based on other similar formulations, e.g., U.S. Pat. No. 4,957,730 (PENLAC® available from Dermik®).

Certain aspects of stabilization of micronized particles in pharmaceutical compositions are addressed in the literature. For example, U.S. Pat. No. 5,858,410 discloses pharmaceutical compositions containing particles of active agents of average diameter less than 5 microns, having been comminuted, without prior conversion into a melt, by using a piston-gap homogenizer. U.S. patent application Ser. No. 10/440,368 discloses the use of a phospholipid surface active agent to stabilize microparticles of solid fenofibrate in an orally administered pharmaceutical composition. U.S. Pat. No. 5,091,188 discloses the use of phospholipids, to prevent coalescence of microcrystalline active agents in injectable pharmaceutical compositions. Examples of disclosed phospholipids include lecithin, phosphatidic acid, phosphatidyl ethanolamine, cholesterol, stearylamine, glycolipids and mono-glycerides.

None of the aforementioned references however, discloses an injectable suspension of Posaconazole, that is stable when subjected to terminal steam sterilization and throughout the shelf life of the product. There is a need for such a formulation as it is desirable to ensure the physical stability of the sterilized end product.

### SUMMARY OF THE INVENTION

The present invention provides formulations of posaconazole that are stable when subjected to terminal steam sterilization. These formulations are useful for the treatment of infections, in particular, an aqueous injectable suspension of posaconazole that is homogenously suspended in vehicle with the aid of a phospholipid. In addition, a thermoprotectant agent is employed to reduce autoclave-induced particle size growth, as well as a buffer system to stabilize the phospholipid during autoclaving or during storage after autoclaving. The formulations provided remain stable after at least 20 minutes of autoclaving at 121° C. and after subsequent storage at 4° C. to 40° C. for at least 6 months.

The present invention provides formulations comprising a suspension of Posaconazole, stabilized by a phospholipid, in a mixture comprising a thermoprotectant, and a buffer system.

In some embodiments, the formulation has been sterilized by autoclaving or by irradiation.

In some embodiments, the buffer system comprises sodium phosphate, which may be provided as sodium phosphate monobasic monohydrate, sodium phosphate dibasic anhydrous, or the combination of the two.

In some embodiments, the buffer system comprises an organic buffer.

In some embodiments, the buffer system comprises at least one of histidine, citric acid, glycine, sodium citrate, ammonium sulfate, or acetic acid.

In some embodiments, the buffer system maintains a pH of about 3.0 to about 9.0 after autoclaving.

In some embodiments, the buffer system maintains a pH of about 6.0 to about 8.0 after autoclaving.

In some embodiments, the buffer system maintains a pH of about 6.4 to about 7.6 after autoclaving.

In some embodiments, the phospholipid comprises a natural phospholipid.

In some embodiments, the phospholipid comprises a synthetic phospholipid.

In some embodiments, the phospholipid comprises a natural phospholipid and a synthetic phospholipid.

In some embodiments, the phospholipid comprises 1-palmitoyl-2-oleoyl-sn-glycero-3-phosphocholine (POPC).

In some embodiments, the thermoprotectant comprises trehalose.

In some embodiments, the phospholipid comprises 1-palmitoyl-2-oleoyl-sn-glycero-3-phosphocholine (POPC), the thermoprotectant comprises trehalose, and the buffer system comprises sodium phosphate monobasic, sodium phosphate dibasic, or the combination of sodium phosphate monobasic and sodium phosphate dibasic, which phosphates may be provided, respectively, as the monohydrate and anhydrous forms.

In some embodiments, the Posaconazole has a particle size distribution whose median value is between about 1.0 and about 8.0 microns, with not more than about 3000 particles of 10 microns or greater size per small volume parenteral unit and not more than about 300 particles of 25 microns or greater size per small-volume parenteral unit.

In some embodiments, the Posaconazole has a particle size distribution whose median value is between about 1.0 and about 5.0 microns, with not more than about 3000 particles of 10 microns or greater size per small volume parenteral unit and not more than about 300 particles of 25 microns or greater size per small-volume parenteral unit.

In some embodiments, the Posaconazole has a particle size distribution whose median value is between about 1.2 and about 4.5 microns, with not more than about 3000 particles of 10 microns or greater size per small volume parenteral unit and not more than about 300 particles of 25 microns or greater size per small-volume parenteral unit.

In some embodiments, the Posaconazole has a particle size distribution whose median value is between about 1.5 and about 3.0 microns, with not more than about 3000 particles of 10 microns or greater size per small volume parenteral unit and not more than about 300 particles of 25 microns or greater size per small-volume parenteral unit.

In some embodiments, the formulation has ingredients comprising:

| Ingredient | Concentration range |
|---|---|
| Posaconazole | about 50 mg/ml |
| POPC | about 40 mg/ml |
| Sodium Phosphate, | about 0.35 mg/ml |
| monobasic, monohydrate, | |
| USP | |
| Sodium Phosphate, dibasic, | about 1.1 mg/ml |
| anhydrous, USP | |
| Trehalose | about 250 mg/ml |
| Water for Injection, USP q.s. | 1 ml |
| ad | |

at a pH of about 7.2 (after autoclaving).

In some embodiments, the formulation has ingredients comprising:

| Ingredient | Concentration range |
|---|---|
| Posaconazole | about 50 mg/ml |
| POPC | about 40 mg/ml |
| Sodium Phosphate, | about 0.04 mg/ml |
| monobasic, monohydrate, | |
| USP | |
| Sodium Phosphate, dibasic, | about 1.4 mg/ml |
| anhydrous, USP | |
| Trehalose | about 250 mg/ml |
| Water for Injection, USP q.s. | 1 ml |
| ad | |

at a pH of about 7.4 (after autoclaving).

In some embodiments, the formulation has ingredients comprising:

| Ingredient | Concentration range |
|---|---|
| Posaconazole | about 1 to about 100 mg/ml |
| POPC | about 10 to about 60 mg/ml |
| Sodium Phosphate, | about 0.01 to about 0.6 mg/ml |
| monobasic, monohydrate, | |
| USP | |
| Sodium Phosphate, dibasic, | about 0.04 to about 1.5 mg/ml |
| anhydrous, USP | |
| Trehalose | about 10 to about 300 mg/ml |
| Water for Injection, USP q.s. | about 1 ml |
| ad | |

In some embodiments, the formulation has ingredients comprising:

| Ingredient | Concentration range |
|---|---|
| Posaconazole | about 40 to about 60 mg/ml |
| POPC | about 20 to about 50 mg/ml |
| Trehalose | about 100 to about 250 mg/ml |
| Water for Injection, USP q.s. | about 1 ml |
| ad | |

In some embodiments, the formulation has ingredients comprising:

| Ingredient | Concentration |
|---|---|
| Posaconazole | about 50 mg/ml |
| POPC | about 40 mg/ml |
| Histidine | about 3 mg/ml |
| Citric acid monohydrate | about 0.24 mg/ml |
| Trehalose | about 250 mg/ml |
| Water q.s. ad | about 1 ml |

at a pH of about 6.4.

In some embodiments, the formulation has ingredients further comprising an antioxidant.

In some embodiments, the antioxidant comprises propyl gallate at a concentration of about 0.02 to about 0.005 mg/ml.

In some embodiments, the antioxidant comprises butylated hydroxytoluene at a concentration of about 0.1 to about 0.02 mg/ml.

In some embodiments, the antioxidant comprises alpha-D-tocopherol at a concentration of about 0.5 to about 0.01 mg/ml.

In some embodiments, the formulation has ingredients comprising:

| Ingredient | Concentration |
|---|---|
| Posaconazole | about 50 mg/ml |
| POPC | about 40 mg/ml |
| Histidine | about 3 mg/ml |
| Citric acid monohydrate | about 0.24 mg/ml |
| Propyl gallate | about 0.01 mg/ml |
| Butylated hydroxytoluene | about 0.05 mg/ml |
| Trehalose | about 250 mg/ml |
| Water q.s. ad | about 1 ml |

at a pH of about 6.4.

In some embodiments, the formulation has ingredients comprising:

| Ingredient | Concentration |
|---|---|
| Posaconazole | about 50 mg/ml |
| POPC | about 40 mg/ml |
| Histidine | about 3 mg/ml |
| Citric acid monohydrate | about 0.24 mg/ml |
| Alpha-D-tocopherol | about 0.05 mg/ml |
| Trehalose | about 250 mg/ml |
| Water q.s. ad | about 1 ml |

at a pH of about 6.5.

In some embodiments, the concentration of posaconazole is about 50 g/L, the concentration of 1-Palmitoyl-2-oleoyl-sn-glycerol-3-phosphocholine (POPC) is about 40 g/L, and the concentration of trehalose is about 250 g/L.

In some embodiments, the concentration of posaconazole is about 1 to about 100 g/L, the concentration of 1-Palmitoyl-2-oleoyl-sn-glycerol-3-phosphocholine (POPC) is about 10 to about 60 g/L, and the concentration of trehalose is about 10 to about 300 g/L.

In some embodiments, the concentration of posaconazole is about 40 to about 60 g/L, the concentration of 1-Palmitoyl-2-oleoyl-sn-glycerol-3-phosphocholine (POPC) is about 20 to about 50 g/L, and the concentration of trehalose is about 100 to about 250 g/L.

In some embodiments, the formulation has a wt. ratio of phospholipid to Posaconazole between about 60:1 and about 1:10.

In some embodiments, the formulation has a wt. ratio of phospholipid to Posaconazole between about 1:1 and about 1:5.

In some embodiments, the formulation has a wt. ratio of phospholipid to Posaconazole between about 1:1 and about 4:5.

In some embodiments, the formulation has a wt. ratio of thermoprotectant to Posaconazole between about 300:1 and about 1:10.

In some embodiments, the formulation has a wt. ratio of thermoprotectant to Posaconazole between about 1:1 and about 6:1.

In some embodiments, the formulation has a wt. ratio of thermoprotectant to phospholipid between about 30:1 and about 1:6.

In some embodiments, the formulation has a wt. ratio of thermoprotectant to phospholipid between about 5:4 and about 30:4.

In some embodiments, the invention encompasses a method of treating or preventing an infection in an animal in need thereof which comprises administering to said animal an effective amount of the formulation. In some embodiments, the animal is a mammal, a bird, a fish, or a reptile.

In some embodiments, the animal is a mammal, including but not limited to a human.

In some embodiments, the infection is caused by a fungus or a parasite.

In some embodiments, the infection is selected from the group consisting of:

oropharyngeal or esophageal candidiasis; refractory oropharyngeal and esophageal candidiasis; invasive aspergillosis, candidiasis, fusariosis, scedosporiosis, infections due to dimorphic fungi, zygomycosis, and invasive infections due to rare molds or yeasts; invasive mycoses in patients who are refractory to, or intolerant of, other therapies; Candidiasis, invasive mould infections in patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation; Chagas disease; and, Leishmaniasis.

In some embodiments, the invention encompasses a method wherein said formulation is administered intravenously.

In some embodiments, the invention encompasses a method wherein said formulation is administered intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracistemally, intravascularly, intraventricularly, intraosseously, via irrigation of infected bone, or via application as part of any admixture with cement for prosthetic devices.

In some embodiments, the formulation further comprises a second active ingredient selected from one or more of the group consisting of: antifungals such as azoles; amphotericin B; deoxycholate amphotericin B; flucytosine; terbinafine; antibacterials; antivirals; steroids; nonsteroidal anti-inflammatory drugs (“NSAIDs”); chemotherapeutics; and anti-emitics.

In some embodiments, the invention encompasses a method further comprising administering a second active ingredient selected from one or more of the group consisting of: antifungals such as azoles; amphotericin B; deoxycholate amphotericin B; flucytosine; terbinafine; antibacterials; antivirals; steroids; nonsteroidal anti-inflammatory drugs (“NSAIDs”); chemotherapeutics; and, anti-emitics.

In some embodiments, the formulation is further characterized by providing at least one of a mean maximum plasma concentration (Cₘₐₓ) of Posaconazole of at least about 147 ng/ml at steady state, and a mean plasma Area Under the Curve over 24 hours (AUC) value of Posaconazole of at least about 3216 ng·hr/ml at steady state, when said formulation is infused over about 1 hour to deliver a dose of at least 50 mg of Posaconazole, and repeated at an interval of about 24 hours.

In some embodiments, the formulation is further characterized by providing at least one of a mean maximum plasma concentration (Cₘₐₓ) of Posaconazole of at least about 467 ng/ml at steady state, and a mean plasma Area Under the Curve over 24 hours (AUC) value of Posaconazole of at least about 9840 ng·hr/ml at steady state, when said formulation is infused over about 1 hour to deliver 100 mg of Posaconazole, and repeated at an interval of once per day.

In some embodiments, the formulation is further characterized by providing at least one of a mean maximum plasma concentration (Cₘₐₓ) of Posaconazole of at least about 852 ng/ml at steady state, and a mean plasma Area Under the Curve over 24 hours (AUC) value of Posaconazole of at least about 24,600 ng·hr/ml at steady state, when said formulation is infused over about 1 hour to deliver 200 mg of Posaconazole, and repeated at an interval of once per day.

In some embodiments, the formulation is further characterized by providing, after administration of a dosage of about 100 mg of said Posaconazole, at least one of: a mean plasma half-life in a range of about 14.9 to about 38.4 hours; and a mean plasma steady state volume of distribution of about 200 to about 500 L.

In some embodiments, the formulation is further characterized by providing at least one of a mean maximum plasma concentration (Cₘₐₓ) of Posaconazole of at least about 1480 ng/ml at steady state, and a mean plasma Area Under the Curve over 24 hours (AUC) value of Posaconazole of at least about 24,600 ng·hr/ml at steady state, when said formulation is infused over about 1 hour to deliver at least 200 mg of Posaconazole, and repeated at an interval of about 24 hours.

In some embodiments, the formulation is further characterized as providing, after administration of a dosage of about 200 mg of said Posaconazole, at least one of: a mean plasma half-life of about 18.7 to about 35.5 hours; and a mean plasma steady state volume of distribution of about 200 to about 500 L.

In some embodiments, the formulation is further characterized as providing, after administration of a dosage of about 400 mg of said Posaconazole, at least one of: a mean plasma half-life of about 18.5 to about 51.4 hours; and a mean plasma steady state volume of distribution of about 200 to about 500 L.

In some embodiments, the formulation is further characterized as providing, after administration of a dosage of about 600 mg of said Posaconazole, at least one of: a mean plasma half-life of about 27.2 to about 50.6 hours; and a mean plasma steady state volume of distribution of about 200 to about 500 L.

In some embodiments, the formulation is further characterized as providing a mean Posaconazole blood concentration profile substantially similar to that of FIG. 1, when said formulation is infused over about 1 hour to deliver 25-600 mg of Posaconazole.

In some embodiments, the formulation is further characterized as providing a mean Posaconazole plasma concentration profile substantially similar to that of FIG. 2, when said formulation is infused over about 1 hour to deliver 25-600 mg of Posaconazole.

In some embodiments, the formulation is further characterized as providing a ratio of mean Posaconazole blood Cₘₐₓ to mean Posaconazole plasma Cₘₐₓ of between about 1.5 and about 3.8, when a single dose of said formulation is infused over about 1 hour to deliver 25-600 mg of Posaconazole.

In some embodiments, the formulation is further characterized as providing a ratio of mean Posaconazole blood Cₘₐₓ to mean Posaconazole plasma Cₘₐₓ of between about 2.1 and about 3.3, when a single dose of said formulation is infused over about 1 hour to deliver 25 mg of Posaconazole.

In some embodiments, the formulation is further characterized as providing a ratio of mean Posaconazole blood Cₘₐₓ to mean Posaconazole plasma Cₘₐₓ of between about 1.9 and about 3.8, when a single dose of said formulation is infused over about 1 hour to deliver 50 mg of Posaconazole.

In some embodiments, the formulation is further characterized as providing a mean Posaconazole blood Cₘₐₓ to mean Posaconazole plasma Cₘₐₓ of between about 2.2 and about 3.3, when a single dose of said formulation is infused over about 1 hour to deliver 100 mg of Posaconazole.

In some embodiments, the formulation is further characterized as providing a ratio of mean Posaconazole blood Cₘₐₓ to mean Posaconazole plasma Cₘₐₓ of between about 1.5 and about 3.2, when a single dose of said formulation is infused over about 1 hour to deliver 200 mg of Posaconazole.

In some embodiments, the formulation is further characterized as providing a ratio of mean Posaconazole blood Cₘₐₓ to mean Posaconazole plasma Cₘₐₓ of between about 1.7 and about 3.3, when a single dose of said formulation is infused over about 1 hour to deliver 400 mg of Posaconazole.

In some embodiments, the formulation is further characterized as providing a ratio of mean Posaconazole blood Cₘₐₓ to mean Posaconazole plasma Cₘₐₓ of between about 1.9 and about 3.1, when a single dose of said formulation is infused over about 1 hour to deliver 600 mg of Posaconazole.

In some embodiments, the formulation is further characterized as providing a ratio of mean Posaconazole blood Cₘₐₓ to mean Posaconazole plasma Cₘₐₓ of between about 1.2 and about 2.5, at steady state when said formulation is infused over about 1 hour to deliver 25-600 mg of Posaconazole, and repeated on a 24-hour basis.

In some embodiments, the formulation is further characterized as providing a ratio of mean Posaconazole blood Cₘₐₓ to mean Posaconazole plasma Cₘₐₓ of between about 1.5 and about 2.3, at steady state when said formulation is infused over about 1 hour to deliver 25 mg of Posaconazole, and repeated on a 24-hour basis.

In some embodiments, the formulation is further characterized as providing a ratio of mean Posaconazole blood Cₘₐₓ to mean Posaconazole plasma Cₘₐₓ of between about 1.5 and about 2.4, at steady state when said formulation is infused over about 1 hour to deliver 50 mg of Posaconazole, and repeated on a 24-hour basis.

In some embodiments, the formulation is further characterized as providing a ratio of mean Posaconazole blood Cₘₐₓ to mean Posaconazole plasma Cₘₐₓ of between about 1.7 and about 2.5, at steady state when said formulation is infused over about 1 hour to deliver 100 mg of Posaconazole, and repeated on a 24-hour basis.

In some embodiments, the formulation is further characterized as providing a ratio of mean Posaconazole blood Cₘₐₓ to mean Posaconazole plasma Cₘₐₓ of between about 1.2 and about 2.0, at steady state when said formulation is infused over about 1 hour to deliver 200 mg of Posaconazole, and repeated on a 24-hour basis.

In some embodiments, the formulation is further characterized as providing a ratio of mean Posaconazole blood Cₘₐₓ to mean Posaconazole plasma Cₘₐₓ of between about 1.2 and about 2.2, at steady state when said formulation is infused over about 1 hour to deliver 400 mg of Posaconazole, and repeated on a 24-hour basis.

In some embodiments, the formulation is further characterized as providing a ratio of mean Posaconazole blood Cₘₐₓ to mean Posaconazole plasma Cₘₐₓ of between about 1.3 and about 1.7, at steady state when said formulation is infused over about 1 hour to deliver 600 mg of Posaconazole, and repeated on a 24-hour basis.

In some embodiments, the formulation has been lyophilized.

In some embodiments, the animal treated is human, while in other embodiments the animal treated is non-human.

In some embodiments, the formulation is one that is bioequivalent to a formulation disclosed herein.

In some embodiments, the method comprises administering to said animal an effective amount of Posaconazole to provide a mean maximum plasma concentration (Cₘₐₓ) of Posaconazole of at least about 467 ng/ml at steady state, and a mean plasma Area Under the Curve over 24 hours (AUC) value of Posaconazole of at least about 9840 ng·hr/ml at steady state, when said formulation is infused over about 1 hour to deliver 100 mg of Posaconazole, and repeated at an interval of about 24 hours.

In some embodiments, the formulation comprises a suspension of posaconazole particles, stabilized by a phospholipid, in a mixture comprising water, a thermoprotectant, and a buffer system, wherein said Posaconazole has a particle size distribution whose particle size median value is between about 1.5 and about 3.0 microns.

In some embodiments, the particle size median value is between about 1.7 and about 2.8 microns.

In some embodiments, the particle size median value is about 2.8 microns.

In some embodiments, the particle size median value is about 2.3 microns.

In some embodiments, the particle size median value is about 1.7 microns.

In some embodiments, the formulation does not have more than about 9 vol % of particles of 1 micron or lesser size.

In some embodiments, the formulation does not have more than about 13 vol % of particles of 1 micron or lesser size.

In some embodiments, the formulation does not have more than about 20 vol % of particles of 1 micron or lesser size.

In some embodiments, the formulation does not have more than about 50 vol % of particles of 1 micron or lesser size.

In some embodiments, the formulation has about 5 to about 25 vol % of particles of 1 micron or lesser size.

In some embodiments, the formulation has about 25 to about 50 vol % of particles of 1 micron or lesser size.

In some embodiments, the formulation does not have more than about 3000 particles of 10 microns or greater size per small-volume parenteral unit and not more than about 300 particles of 25 microns or greater size per small-volume parenteral unit.

In some embodiments wherein said phospholipid is 1-Palmitoyl-2-oleoyl-sn-glycerol-3-phosphocholine (POPC) and said thermoprotectant is Trehalose.

In some embodiments the formulation has at least the following ingredients:

| | Amount |
|---|---|
| Ingredients | (g/Liter) |
| Posaconazole | about 50 |
| Micronized, Parenteral grade | |
| 1-Palmitoyl-2-oleoyl-sn-glycerol-3- | about 40 |
| phosphocholine, Powder (POPC) | |
| Trehalose | about 250. |

In some embodiments, the particle size median value is between about 1.5 and about 3.0 microns after at least 6 months of storage at 25° C., or after at least 24 months of storage at 4° C., wherein said storage occurs after said formulation has been terminally sterilized by autoclaving at 121° C. for up to 20 minutes.

In some embodiments, the particle size median value is between about 1.5 and about 3.0 microns after being terminally sterilized by autoclaving at 121° C. for up to 150 minutes.

In some embodiments, the particle size median value is between about 1.5 and about 3.0 microns after being subjected to one 20-minute autoclave cycle at 121° C. and up to five additional 30-minutes autoclave cycles at 121° C., for a cumulative exposure at 121° C. of up to 170 minutes.

In some embodiments, the formulation further comprises a second active ingredient selected from the group consisting of antifungals, flucytosine, terbinafine, antibacterials, antivirals, steroids, nonsteroidal anti-inflammatory drugs (“NSAIDs”), chemotherapeutics, and anti-emitics.

In some embodiments, said second active ingredient is an antifungal selected from the group consisting of: flucytosine, terbinafine, amphotericin B; deoxycholate amphotericin B.

In some embodiments, the formulation is further characterized by providing at least one of a mean maximum plasma concentration (Cₘₐₓ) of Posaconazole of at least about 1080 ng/ml at steady state, and a mean plasma Area Under the Curve over 24 hours (AUC) value of Posaconazole of at least about 20,100 ng·hr/ml at steady state, after said formulation is infused over about 1 hour to deliver 100 mg of Posaconazole, and said infusion is repeated at an interval of once per day.

In some embodiments, the formulation is further characterized by providing at least one of a mean maximum plasma concentration (Cₘₐₓ) of Posaconazole of at least about 2030 ng/ml at steady state, and a mean plasma Area Under the Curve over 24 hours (AUC) value of Posaconazole of at least about 38,100 ng·hr/ml at steady state, after said formulation is infused over about 1 hour to deliver 200 mg of Posaconazole, and said infusion is repeated at an interval of once per day.

In some embodiments, the formulation is further characterized by providing at least one of a mean maximum plasma concentration (Cₘₐₓ) of Posaconazole of at least about 2820 ng/ml at steady state, and a mean plasma Area Under the Curve over 24 hours (AUC) value of Posaconazole of at least about 53,100 ng·hr/ml at steady state, after said formulation is infused over about 1 hour to deliver 300 mg of Posaconazole, and said infusion is repeated at an interval of once per day.

In some embodiments, the formulation is further characterized by providing at least one of a mean maximum plasma concentration (Cₘₐₓ) of Posaconazole of at least about 3830 ng/ml at steady state, and a mean plasma Area Under the Curve over 24 hours (AUC) value of Posaconazole of at least about 75,400 ng·hr/ml at steady state, after said formulation is infused over about 1 hour to deliver 400 mg of Posaconazole, and said infusion is repeated at an interval of once per day.

In some embodiments, the formulation is further characterized by providing at least one of a mean plasma half-life of about 36.8 hours and a mean plasma steady state volume of distribution of about 334 L, after said formulation is infused over about 1 hour to deliver 100 mg of Posaconazole, and said infusion is repeated at an interval of once per day.

In some embodiments, the formulation is further characterized by providing at least one of a mean plasma half-life of about 38.6 hours and a mean plasma steady state volume of distribution of about 339 L, after said formulation is infused over about 1 hour to deliver 200 mg of Posaconazole, and said infusion is repeated at an interval of once per day.

In some embodiments, the formulation is further characterized by providing at least one of a mean plasma half-life of about 33.3 hours and a mean plasma steady state volume of distribution of about 348 L, after said formulation is infused over about 1 hour to deliver 400 mg of Posaconazole, and said infusion is repeated at an interval of once per day.

In some embodiments, the formulation is further characterized as providing at least one of a mean Posaconazole steady state plasma concentration profile substantially similar to that of the 100 mg curve of FIG. 7, after said formulation is infused over about 1 hour to deliver 100 mg of Posaconazole and said infusion is repeated at an interval of once per day.

In some embodiments, the formulation is further characterized as providing a mean Posaconazole steady state plasma concentration profile substantially similar to that of the 200 mg curve of FIG. 7, after said formulation is infused over about 1 hour to deliver 200 mg of Posaconazole and said infusion is repeated at an interval of once per day.

In some embodiments, the formulation is further characterized as providing a mean Posaconazole steady state plasma concentration profile substantially similar to that of the 400 mg curve of FIG. 7, after said formulation is infused over about 1 hour to deliver 400 mg of Posaconazole and said infusion is repeated at an interval of once per day.

In some embodiments, the formulation is further characterized as providing a mean Posaconazole plasma trough (Cₘₐₓ) profile substantially similar to that of the 100 mg curve of FIG. 8, after said formulation is infused over about 1 hour to deliver 100 mg of Posaconazole and said infusion is repeated at an interval of once per day.

In some embodiments, the formulation is further characterized as providing a mean Posaconazole plasma trough (Cₘᵢₙ) profile substantially similar to that of the 200 mg curve of FIG. 8, after said formulation is infused over about 1 hour to deliver 200 mg of Posaconazole and said infusion is repeated at an interval of once per day.

In some embodiments, the formulation is further characterized as providing a mean Posaconazole plasma trough (Cₘᵢₙ) profile substantially similar to that of the 400 mg curve of FIG. 8, after said formulation is infused over about 1 hour to deliver 400 mg of Posaconazole and said infusion is repeated at an interval of once per day.

In some embodiments, the formulation is further characterized as providing a mean Posaconazole plasma concentration profile substantially similar to that of the intravenous curve of FIG. 9, after said formulation is infused over about 1 hour to deliver 100 mg of Posaconazole.

In some embodiments, the formulation is administered by first administering an intravenous loading dose and then administering a maintenance dose.

In some embodiments, the loading dose is about 200 to about 400 mg. and said maintenance dose is an intravenous dose of about 100 mg/day to about 400 mg/day.

In some embodiments, the method further comprises the step of administering Posaconazole oral suspension at a maintenance dose of about 100 mg/day to about 800 mg/day as a single or divided dose.

In some embodiments, the formulation comprises a suspension of posaconazole particles, stabilized by 1-Palmitoyl-2-oleoyl-sn-glycerol-3-phosphocholine (POPC) in a mixture comprising water, trehalose, and a buffer system, wherein said posaconazole has a particle size distribution whose particle size median value is between about 1.5 and about 3.0 microns, and wherein the concentration of posaconazole is about 50 g/L, the concentration of 1-Palmitoyl-2-oleoyl-sn-glycerol-3-phosphocholine (POPC) is about 40 g/L, and the concentration of trehalose is about 250 g/L.

### BRIEF DESCRIPTION OF THE DRAWINGS

FIG. 1 shows Posaconazole mean blood concentration-time profiles in healthy volunteers after 1 hr intravenous infusions of 25, 50, 100, 200, 400, and 600 mg Posaconazole.

FIG. 2 shows Posaconazole mean plasma concentration-time profiles in healthy volunteers after 1 hr intravenous infusions of 25, 50, 100, 200, 400, and 600 mg Posaconazole.

FIG. 3 shows Posaconazole mean plasma and blood concentration-time profiles in healthy volunteers after 1 hr intravenous infusion of 25 mg Posaconazole.

FIG. 4 shows Posaconazole mean plasma and blood concentration-time profiles in healthy volunteers after 1 hr intravenous infusion of 600 mg Posaconazole.

FIG. 5 shows mean plasma concentration-time profiles of Posaconazole in male monkeys following single intravenous administration of 8 mg/kg Posaconazole in various formulations.

FIG. 6A shows mean Cₘₐₓ of Posaconazole in male monkeys following single intravenous administration of 8 mg/kg Posaconazole in various formulations.

FIG. 6B shows AUC (1) of Posaconazole in male monkeys following single intravenous administration of 8 mg/kg Posaconazole in various formulations.

FIG. 7 shows mean POS steady-state plasma concentration-time profile (on study day 14 after 10 consecutive daily doses) following a multiple daily-dose IV infusion of 100, 200, or 400 mg POS IV to healthy male volunteers.

FIG. 8 shows mean POS plasma trough (Cₘᵢₙ) values after a multiple daily-dose IV infusion of 100, 200, or 400 mg POS IV to healthy male volunteers (study days 10 to 14 correspond with 6 to 10 consecutive daily doses).

FIG. 9 shows mean POS plasma concentration-time profile in healthy male volunteers after a single-dose IV infusion of 100 mg POS IV or a single 100-mg dose of POS oral.

### DETAILED DESCRIPTION OF THE INVENTION

The present invention encompasses formulations suitable for parenteral administration, e.g., by injection, for treating an infection. These formulations comprise a suspension of Posaconazole particles, stabilized by a phospholipid, in a mixture comprising water, a thermoprotectant, and a buffer system. The inventors have discovered that the Posaxonazole particle size distribution of the suspension greatly affects the pharmacokinetic characteristics of the administered formulation. Since Posaconazole is minimally soluble in water, a suspension formulation is advantageous. Phospholipids have been found to be effective surfactants in forming stable suspensions of Posaconazole in water or an aqueous medium.

These phospholipids can degrade when subjected to the temperature excursions experienced during terminal sterilization (e.g., autoclaving), a step which is necessary to assure the sterility of any injectable formulation. Thus, a thermoprotectant is used to reduce agglomeration and crystal growth of the Posaconazole particles during autoclaving. Parenteral buffer systems are typically designed to be at physiological pH of about 7.4. Phospholipids are known to be stable at a pH range of about 6 to about 7. Furthermore, pH adjustment of injectable formulations can be necessary to achieve physiological compatibility, and thus, for example, to minimize injection-site irritation. In addition, the rate of phospholipid hydrolysis can be temperature-sensitive. Thus, in the present formulations, the buffer systems are designed to meet physiological pH requirements, and to maintain the temperature/pH-dependent chemical stability of the phospholipid in the formulation during high temperature excursions (such as experienced during autoclaving), and throughout shelf life.

In accordance with the above, it was found that POPC, an ingredient that acts as a suspension stabilizer, was sensitive to autoclaving. Certain buffer systems were found to control degradation of POPC-containing Posaconazole formulations during autoclaving. For example, such formulations were found to be stable after at least 20 minutes of autoclaving at 121° C. In addition, these buffer systems stabilize such formulations during storage at 4° C. for at least 24 months following 20 minutes of autoclaving, and at 25° C. for at least 6 months. Similarly, other phospholipids that are similar to POPC could be used to stabilize the formulations disclosed herein. For example, unsaturated phospholipids with an acyl chain length ranging from C₁₂ to C₂₀ wherein the degree of unsaturation of the acyl chain ranges from 1 to 4; as well as saturated phospholipids with an acyl chain length ranging from Cl₂ to C₁₈ are useful according to the present invention.

Examples of useful unsaturated phospholipids include:
1-palmitoyl-2-oleoyl-sn-glycero-3-phosphocholine (“POPC”),
1,2-Myristoleoyl-sn-Glycero-3-Phosphocholine

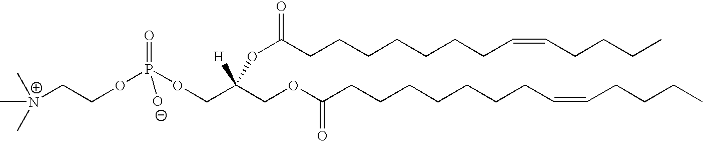

1,2-Palmitoleoyl-sn-Glycero-3-Phosphocholine

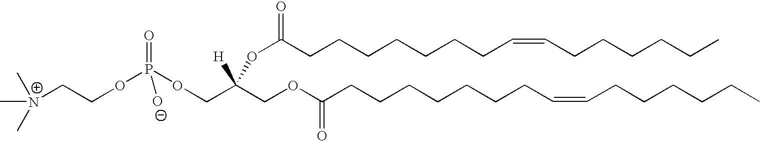

1,2-Dioleoyl-sn-Glycero-3-Phosphocholine (DOPC)

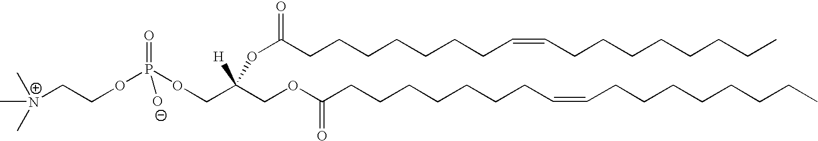

1,2-Dioleoyl-sn-Glycero-3-Phosphoethanolamine (DOPE)

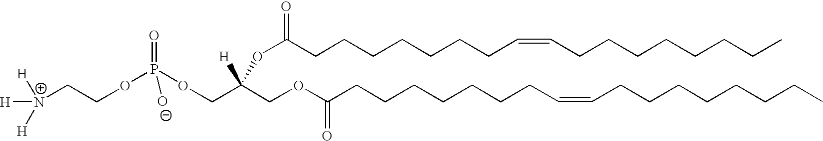

1,2-Linoleoyl-sn-Glycero-3-Phosphocholine

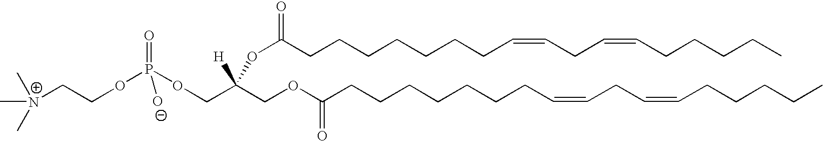

and
1-Oleoyl-2-Myristoyl-sn-Glycero-3-Phosphocholine

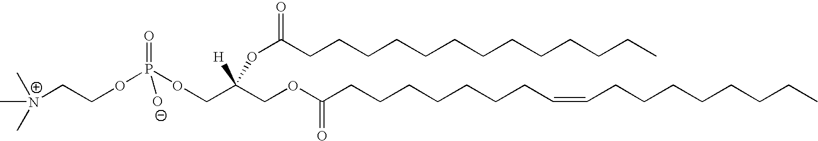

or combinations thereof.

Examples of saturated phospholipids include:
1,2-Dilauryl-sn-Glycero-3-Phosphocholine (DLPC)

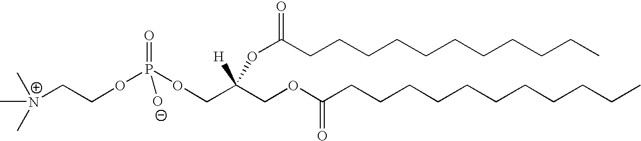

1,2-Dimyristoyl-sn-Glycero-3-Phosphocholine (DMPC)

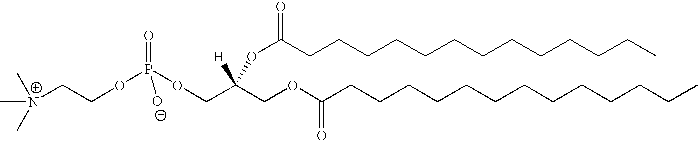

1,2-Dipalmitoyl-sn-Glycero-3-Phosphocholine (DPPC)

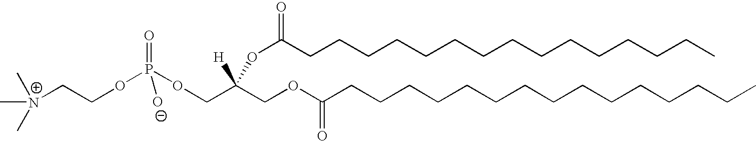

and
1,2-Stearoyl-sn-Glycero-3-Phosphocholine (DSPC)

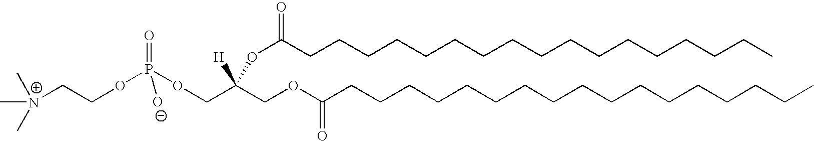

or combinations thereof.

Unsaturated phospholipids are known be to prone to oxidation. To prevent such oxidation, an antioxidant can be employed. In some embodiments, the antioxidant comprises propyl gallate, preferably at a concentration of about 0.02 to about 0.005 mg/ml. In other embodiments, the antioxidant comprises butylated hydroxytoluene, preferably at a concentration of about 0.1 to about 0.02 mg/ml. In related embodiments, the antioxidant comprises propyl gallate, preferably at a concentration of about 0.02 to about 0.005 mg/ml, in combination with butylated hydroxytoluene, preferably at a concentration of about 0.1 to about 0.02 mg/ml. In yet other embodiments, the antioxidant comprises alpha-D-tocopherol, preferably at a concentration of about 0.5 to about 0.01 mg/ml.

The inventors have found certain ratios of components to result in advantageous formulations. For example, the weight ratio of phospholipid to Posaconazole is preferably between about 1:0.1 and about 1:10, more preferably, between about 1:1 and about 1:5, still more preferably, between about 1:1 and about 4:5. The weight ratio of thermoprotectant to Posaconazole is preferably between about 0.5:1 and about 6:1, more preferably, between about 2:1 and about 6:1. The weight ratio of thermoprotectant to phospholipid is preferably, between about 20:1 and about 5:4, more preferably, between about 20:4 and about 30:4.

The posaconazole injectable suspensions described herein can be prepared by a process that includes mixing, high pressure homogenization (or other particle size reduction technology), filtration, filling and terminal steam sterilization.

The manufacturing process includes a premix step in which the drug substance, phospholipid, buffer salts, and water are mixed together and then continuously circulated through the high pressure homogenizer until the desired particle size and particle size distribution is reached. Parameters that impact the high pressure homogenization include (but are not limited to) pressure, flow rate, temperature, and number of passes through the system, and can be adjusted to vary the particle size distribution of the product. After high pressure homogenization is complete, the premix is transferred to a larger compounding vessel where the thermoprotectant (e.g., trehalose) is added. Particularly, the inventors have found that the addition of the thermoprotectant after the completion of the high pressure homogenization is preferable in controlling the particle size of the Posaconazole particles during autoclaving and subsequent storage. This is believed to be due to the ability of the thermoprotectant to further stabilize the phospholipid structure as a result of the higher osmolality outside the phospholipid structure relative to the osmolality within the phospholipid structure, when added after high pressure homogenization. The suspension is pH adjusted (if needed) by adding, e.g., sodium hydroxide or phosphoric acid, and further mixed and then filtered through a 10 micron filter, filled and sealed. Filters of different pore size ratings may be used to further adjust the particle size and particle size distribution of the product prior to filling and sealing. After filling is complete, the suspension is terminally sterilized for at least 20 minutes in a steam autoclave at about 120° C. (preferably 121.5±0.5° C.].

Particle sizes are herein characterized on a volume-weighted basis, typically by laser diffraction particle size analysis. Analyzers such as those manufactured by Malverne®, Sympatic®, and Horibe® have been used to herein provide data such as median particle sizes and volume percent of particles below a stated particle size, e.g., 1 micron.

The formulations of the present invention comprise a suspension of solid particles of Posaconazole of specific particle size distribution in an aqueous phase. The particle size distribution displayed in the suspended particles is critical for physiological compatibility, syringeability, physical stability of the suspension, re-suspendability, and for pharmacokinetic characteristics and bio-distribution (i.e., sequestration within specific bodily tissues). Since these characteristics are critical to the formulation as delivered to the patient, it is important that processes that contribute to changes in particle size distribution after micronization are controlled.

Such processes can include agglomeration during autoclaving, and de-suspension due to temperature excursions and/or agitation experienced during shipping and storage. It is the particle size distribution in the formulation as ready for administration to the patient that influences pharmacokinetic characteristics and bio-distribution.

The inventors of the present invention have determined that for injectable formulations of Posaconazole, these characteristics are brought within advantageous ranges with particle size distributions whose median values are between about 1.0 to about 8.0 microns, preferably, between about 1.0 to about 5.0 microns, more preferably between about 1.2 to about 4.5 microns, or still more preferably between about 1.5 to about 3.0 microns. A particularly preferable range of median particle sizes is between about 1.7 and about 2.8 microns. In each case, the particle size distributions display not more than about 3000 particles of 10 microns or greater size per small volume parenteral unit and not more than about 300 particles of 25 microns or greater size per small volume parenteral unit. In some preferred embodiments, the volume percent of particles of 1 micron or lesser size is less than about 50%. In some of these embodiments, this population of particles is less than about 25%, or even less than 10%.

In the injectable formulations of the present invention, which include POPC, it has been found useful to maintain a pH range of between about 3.0 and about 9.0, preferably between about 6.0 and about 8.0, and more preferably between about 6.4 and about 7.6.

The inventors have found that certain organic buffers, e.g., histidine and citric acid, are more advantageous in controlling the pH-related degradation of POPC in the formulation. Components used in pH adjustment systems can also function as components of the buffer system, after pH adjustment has been achieved. Non-limiting examples of pH adjustment system components that function in this way include sodium hydroxide, hydrochloric acid, and phosphoric acid.

### Anti-Infective Applications

The present invention encompasses methods of prevention and treatment of a variety of infections caused by a broad spectrum of infectious agents. The term “infection” is understood to include, but not be limited to, those disease states caused by molds, yeasts and other infectious agents, such as: Candida, dermatophytes, Dimorphics, Dematiaceous (e.g., Alternaria and Bipolaris), Aspergillus, Acremonium, Basidiomycetes, Bjerkandera, Coprinus, Paecilomyces, Microsporum, Trichophyton, Pseudallescheria, Schizophyllum, Crytococcus, Histoplasma, Blastomyces, Coccidioides, Fusarium, Exophiala, Zygomycocetes (e.g., Mucor, Rhizopus, and Rhizomucor), Kluyveromyces, Saccharomyces, Yarrowia, Pichia, Epidermophyton, Paracoccidioides, Scedosporium, Apophysomyces, Curvularia, Penicillium, Fonsecaea, Wangiella, Sporothrix, Pneumocystis, Trichosporon, Absidia, Cladophialophora, Ramichloridium, Syncephalastrum, Madurella, Scytalidium, Leshmania, protozoa, bacteria, gram negatives, gram positives, anaerobes, including Legionella Borrelia, Mycoplasma, Treponema, Gardneralla, Trichomononas and Trypanosoma.

The present invention is intended to treat both opportunistic and non-opportunistic infections, where the term “opportunistic” as used herein denotes those infections caused by organisms capable of causing a disease only in a host whose resistance is lowered, e.g., by chemotherapy or H.I.V.

In particular, Posaconazole is useful in the prevention and/or treatment of the following disease states:
Initial (first line) treatment of oropharyngeal or esophageal candidiasis; Salvage therapy of azole-refractory oropharyngeal and esophageal candidiasis (e.g., in patients who have failed oral fluconazole and/or itraconazole);
Initial treatment of invasive aspergillosis, candidiasis, fusariosis, scedosporiosis, infections due to dimorphic fungi (e.g., cryptococcosis, coccidioidomycosis, paracoccidioidomycosis, histoplasmosis, blastomycosis), zygomycosis, and invasive infections due to rare moulds and yeasts;
Salvage therapy for invasive mycoses in patients who are refractory to or intolerant of other therapies (e.g., amphotericin B, lipid formulations of amphotericin B, caspofungin, voriconazole and/or itraconazole);
Prevention of invasive Candidiasis, invasive mould infections (including zygomycosis and aspergillosis) in patients at high risk, including patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation; Chagas disease (Trypanosomiasis due to T. cruzi) including acute and chronic forms; and, Leishmaniasis, including visceral and localized forms.

### Administration

Immuno-suppressant therapy (e.g., chemotherapy, radiation therapy, myeloablative conditioning regimens) often results in one or more of the above-referenced infections. The present invention encompasses the administration of a Posaconazole formulation adjunctive to immuno-suppressant therapy, wherein the Posaconazole formulation functions prophylactically with regard to opportunistic infections including the above-referenced disease states.

The present invention encompasses a variety of modes of administration to any part, organ, interstice or cavity of an animal's body that is subject to an infection. A non-limiting set of examples of modes by which the posaconazole formulations of the present invention may be administered includes: intravenously, intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracisternally, intravascularly, intraventricularly, intraosseously, via irrigation of infected bone, and via application as part of any admixture with cement for prosthetic devices.

Co-formulations comprising combinations of Posaconazole and at least one other active ingredient are also within the scope of the present invention. Non-limiting examples of such active ingredients include: antifungals such as echinocandins (including caspofungin, micafungin, and anidulafungin) and azoles (including voriconazole, itraconazole, fluconazole, ketoconazole, ravuconazole); amphotericin B; deoxycholate amphotericin B; flucytosine; and terbinafine.

Also within the scope of this invention are combinations with an antibacterial, antiviral, steroid, or nonsteroidal anti-inflammatory drugs (“NSAIDs”), chemotherapeutics, and/or anti-emitics. Similarly, co-administration of Posaconazole with at least one of the above active ingredients, aside from within a single formulation, is also within the scope of the present invention.

Also within the scope of the present invention are a variety of dosing regimens, each consisting of a frequency of dosing and a duration of administration. Preferred frequencies of dosing include once every 12, 24, 36 and 48 hours. Preferred durations of administration are within the range of 30 minutes to 4 hours, more preferably, 1 to 2 hours. Also included within the scope of preferred administration is bolus dosing, at various rates and various doses, and combinations of a bolus loading dose, or several bolus loading doses, with an intravenous infusion maintenance dose that provides therapeutic plasma concentration ranges similar to or exceeding those described in Table 18 and elsewhere infra.

As used herein, the following terms shall have the definitions set forth below.

As used herein, the phrase “small-volume parenteral unit” refers to single-dose or multiple-dose small-volume injection labeled as, or actually containing 100 mL or less.

As used herein, the phrase “phospholipid” refers to a lipid compound that yields on hydrolysis phosphoric acid, an alcohol, fatty acid and a nitrogenous base. Examples include natural and synthetic phoshpholipids, which include lecithin, cephalin, sphingomyelin and 1-palmitoyl-2-oleoyl-sn-glycero-3-phosphocholine (“POPC”).

As used herein, the phrase “natural phospholipid” refers to a phospholipid occurring in nature, or derived from a natural source. Non-limiting examples of natural phospholipids include egg phospholipids, soy phospholipids, and animal tissue phospholipids. Combinations of more than one natural phospholipid are within the scope of the present invention.

As used herein, the phrase “synthetic phospholipid” refers to a man-made phospholipid. Non-limiting examples of synthetic phospholipids include 1-palmitoyl-2-oleoyl-sn-glycero-3-phosphocholine (POPC), 1,2-oleoyl-sn-glycero-3-phosphocholine (DOPC), 1,2-Dilauryl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC) and 1,2-Stearoyl-sn-Glycero-3-Phosphocholine (DSPC). Combinations of more than one synthetic phospholipid are within the scope of the present invention.

As used herein, the phrase “buffer system” refers to a buffer comprising one or more components that maintains a particular pH range. Non-limiting examples of suitable buffer systems include: phosphoric acid; glycine; sodium citrate; histidine; citric acid; acetic acid; tromethamine; ammonium sulfate; and combinations thereof. The aforementioned components are understood to include the salts, hydrates and solvates thereof. Thus, for example, phosphoric acid includes the sodium phosphate or potassium phosphate salts, among other salts. Preferred buffer systems include sodium phosphate monobasic, sodium phosphate dibasic, or a combination thereof. More preferred buffer systems include sodium phosphate monobasic monohydrate, sodium phosphate dibasic anhydrous, or a combination thereof. As used herein, the phrase “organic buffer” refers to a buffer comprising at least one organic compound. Non-limiting examples of suitable organic buffers include: glycine; sodium citrate; histidine; citric acid; acetic acid; and combinations thereof.

As used herein, the term “antioxidant” refers to an agent that hinders oxidation. Exemplary antioxidants include propyl gallate, butylated hydroxytoluene, and alpha-D-tocopherol.

As used herein, the phrase “median particle size” refers to the particle size present in the volume-weighted 50^{th} percentile, as ascertained by laser diffraction particle size analysis such as that performed using analyzers such as Malvern®, Sympatec®, or Horibe®. Particle sizes are measured periodically, and at the termination of, the shelf life, typically up to 24 months after manufacture, when held at either refrigerated or room temperatures. Particle sizes are also measured and maintained when the formulation is diluted into large volume parenterals, e.g., 5% dextrose or water for injection.

As used herein, the phrase “initial median particle size” refers to the particle size present within 1 week after a specified timepoint. For example, the initial median particle size after autoclaving refers to the median particle size present within 1 week after autoclaving has been completed.

As used herein, the term “autoclaving” refers to sterilization by the terminal steam sterilization method. For example, autoclaving for 20 minutes at 121° C. suffices to sterilize the Posaconazole formulations disclosed herein.

As used herein, the phrase “thermoprotectant” refers to an agent that stabilizes the formulation during temperature excursions. In the present invention, a thermoprotectant is used to preserve the phospholipid, which is necessary to control crystal growth and aggolomeration of the Posaconazole particles during autoclaving and subsequent storage. Thermoprotectants are typically water soluble polyhydroxyl compounds. For example, trehalose is a thermoprotectant agent that may be used in conjunction with Posaconazole. Others include maltose, sorbitol, dextrose, sucrose, lactose and mannitol.

As used herein, the term “solvate” refers to a physical association between a compound with one or more solvent molecules. This physical association involves varying degrees of ionic and/or covalent bonding, including hydrogen bonding. In certain instances, the solvate will be capable of isolation, for example, when one or more solvent molecules are incorporated in the crystal lattice of the crystalline solid. The term “solvate” encompasses both solution-phase and isolatable solvates. Non-limiting examples of suitable solvates include hydrates, ethanolates, and methanolates.

As used herein, the term “injectable” means adapted to parenteral administration.

As used herein, the term “fungus” means one of the diverse morphologic forms of yeasts and molds. Fungi include Candida, dermatophytes, Dimorphics, Dematiaceous (e.g., Alternaria and Bipolaris), Aspergillus, Acremonium, Basidiomycetes, Bjerkandera, Coprinus, Paecilomyces, Microsporum, Trichophyton, Pseudallescheria, Schizophyllum, Crytococcus, Histoplasma, Blastomyces, Coccidioides, Fusarium, Exophiala, Zygomycocetes (e.g., Mucor, Rhizopus, and Rhizomucor), Kluyveromyces, Saccharomyces, Yarrowia, Pichia, Epidermophyton, Paracoccidioides, Scedosporium, Apophysomyces, Curvularia, Penicillium, Fonsecaea, Wangiella, Sporothrix, Pneumocystis, Trichosporon, Absidia, Cladophialophora, Ramichloridium, Syncephalastrum, Madurella, Scytalidium, Leshmania, gram negatives, gram positives, Mycoplasma, Treponema, Gardneralla, and Trichomononas.

As used herein, the term “Dematiaceous” means dark conidia and/or hyphae, and includes as non-limiting examples Alternaria, and Bipolaris. Phaeohyphomycosis is an example of a Dematiaceous fungal infection.

As used herein, the term “Zygomycocete” means a class of fungi characterized by sexual reproduction resulting in the formation of zygospore, and asexual reproduction by means of nonmotile spores called sporangiospores or conidia, and includes as non-limiting examples Mucor, Rhizopus, and Rhizomucor.

As used herein, the term “anaerobe” means a microorganism that can live and grow in the absence of oxygen, and includes as non-limiting examples Legionella Borrelia, Mycoplasma, Treponema, Gardneralla, and Trichomononas.

As used herein, the term “parasite” means an organism that lives on or in another and draws its nourishment therefrom. Parasites include Leshmania and Trypansoma, among others.

As used herein, the term “antifungal” means an agent having activity against one or more fungi, and includes echinocandins such as caspofungin, micafungin, and anidulafungin.

As used herein, the term “azole” means divinylenimine, and includes voriconazole, itraconazole, fluconazole, ketoconazole, ravuconazole.

As used herein, the term “mean maximum concentration (Cₘₐₓ)” when followed by the term “at steady state” means that mean maximum concentration value that occurs after administration of a sufficient number of repeated doses of the formulation to generate maximum blood or plasma concentrations that are substantially equivalent to one another in value. Thus, the subsequent maximum concentration values are no longer rising, but rather each peak achieves substantially the same maximum value as the previous one and the next one. Some of the steady state data provided herein were obtained after 10 consecutive daily doses, but the data indicate that steady states may be obtained after as few as 6 daily doses.

As used herein, the term “Cₘᵢₙ” means minimum plasma concentration at the end of the dosing interval.

As used herein, the term “Tₘₐₓ” means the time of maximum plasma concentration.

As used herein, the term “t_{1/2}” means terminal phase half-life.

As used herein, the term “AUC” means the area under the plasma concentration versus time curve.

As used herein, the term “CL” means total body clearance.

As used herein, the term “animal” is understood to include humans, non-human mammals, fish, birds and reptiles.

As used herein, the term “bioequivalent” is understood as having that meaning assigned to the term by the U.S. Food & Drug Administration.

“Bioequivalence means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.” 21 CFR 320.1 (e). Methodologies for determining bioequivalence are given in “Guidance for Industry: Statistical Approaches to Establishing Bioequivalence,” U.S. Department of Health and Human Services, Food and Drug Administration, Center for Drug Evaluation and Research (CDER) June, 2001.

### EXAMPLES

The following non-limiting examples illustrate certain aspects of the invention.

Exemplary formulations of Posaconazole in conjunction with POPC and trehalose using various buffer systems are detailed below in Tables 1-3. These formulations provide ranges for buffer systems that maintain a particular pH range after autoclaving.

TABLE 1
Representative Posaconazole formulations at a pH range of 6.4-7.4

| Function | Ingredient | Concentration range | |
|---|---|---|---|
| Active | Posaconazole | 50 | mg/ml |
| Stabilizer | POPC | 40 | mg/ml |
| Buffer | Glycine | 3.5-10.5 | mg/ml |
| Buffer | Sodium citrate dihydrate | 4-10.2 | mg/ml |
| Buffer | Citric acid monohydrate | 0.01-0.02 | mg/ml |
| Stabilizer | Trehalose | 250 | mg/ml |
| Solvent | Water q.s. ad | 1 | ml |

TABLE 2
Representative Posaconazole formulations at a pH range of 6.4-6.6

| Function | Ingredient | Concentration range | |
|---|---|---|---|
| Active | Posaconazole | 50 | mg/ml |
| Stabilizer | POPC | 40 | mg/ml |
| Buffer | Glycine | 1.5-4.5 | mg/ml |
| Buffer | Citric acid monohydrate | 0.12-0.36 | mg/ml |
| Stabilizer | Trehalose | 250 | mg/ml |
| Solvent | Water q.s. ad | 1 | ml |

TABLE 3
Representative Posaconazole formulations at a pH range of 6.6-6.8

| Function | Ingredient | Concentration range | |
|---|---|---|---|
| Active | Posaconazole | 50 | mg/ml |
| Stabilizer | POPC | 40 | mg/ml |
| Buffer | Histidine | 1.5-4.5 | mg/ml |
| Buffer | Ammonium sulfate | 1-3 | mg/ml |
| Buffer | Hydrochloric acid | 0.1-0.3 | mg/ml |
| Stabilizer | Trehalose | 250 | mg/ml |
| Solvent | Water q.s. ad | 1 | ml |

An exemplary Posaconazole formulation for each of the buffer systems described in Tables 1-3 is provided in Examples 1-3, respectively.

### EXAMPLE 1

| Ingredient | Concentration | |
|---|---|---|
| Posaconazole | 50 | mg/ml |
| POPC | 40 | mg/ml |
| Glycine | 7 | mg/ml |
| Sodium citrate dehydrate | 8 | mg/ml |
| Trehalose | 250 | mg/ml |
| Water q.s. ad | 1 | ml |

Of note, the pH is 7.4 in Example 1.

### EXAMPLE 2

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Trehalose | 250 | mg/ml |
| Water q.s. ad | 1 | ml |

Of note, the pH is 6.4 in Example 2.

### EXAMPLE 3

| Ingredient | Concentration | |
|---|---|---|
| Posaconazole | 50 | mg/ml |
| POPC | 40 | mg/ml |
| Histidine | 3 | mg/ml |
| Ammonium sulfate | 2 | mg/ml |
| Hydrochloric acid | 0.2 | mg/ml |
| Trehalose | 250 | mg/ml |
| Water q.s. ad | 1 | ml |

Of note, the pH is 6.6 in Example 3.

In addition, exemplary Posaconazole formulations that include antioxidant are described in Examples 4-6.

### EXAMPLE 4

| Ingredient | Concentration | |
|---|---|---|
| Posaconazole | 50 | mg/ml |
| POPC | 40 | mg/ml |
| Glycine | 7 | mg/ml |
| Sodium citrate dehydrate | 8 | mg/ml |
| Propyl gallate | 0.01 | mg/ml |
| Butylated hydroxytoluene | 0.05 | mg/ml |
| Trehalose | 250 | mg/ml |
| Water q.s. ad | 1 | ml |

Of note, the pH is 7.4 in Example 4.

### EXAMPLE 5

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Propyl gallate | 0.01 | mg/ml |
| Butylated hydroxytoluene | 0.05 | mg/ml |
| Trehalose | 250 | mg/ml |
| Water q.s. ad | 1 | ml |

The pH is 6.4 in Example 5.

### EXAMPLE 6

| Ingredient | Concentration | |
|---|---|---|
| Posaconazole | 50 | mg/ml |
| POPC | 40 | mg/ml |
| Histidine | 3 | mg/ml |
| Citric acid monohydrate | 0.24 | mg/ml |
| Alpha-D-tocopherol | 0.05 | mg/ml |
| Trehalose | 250 | mg/ml |
| Water q.s. ad | 1 | ml |

The pH is 6.5 in Example 6.

Example 7 is a preferred embodiment of the present invention. This preferred formulation was utilized in a study of the effects of particle size distribution of the final product on the pharmacokinetics of intravenous Posaconazole in monkeys, described below under the heading “Particle Size PK Study.”

### EXAMPLE 7

| Ingredient | Concentration | |
|---|---|---|
| Posaconazole | 50 | mg/ml |
| POPC | 40 | mg/ml |
| Sodium Phosphate, Monobasic, | 0.345 | mg/ml |
| Monohydrate, USP | | |
| Sodium Phosphate, Dibasic, | 1.065 | mg/ml |
| Anhydrous, USP | | |
| Trehalose | 250 | mg/ml |
| Sodium Hydroxide, NF (1.0 N) | for pH adjustment | |
| Phosphoric Acid, NF (20% w/w) | for pH adjustment | |
| Water for injection, USP | qs 1 | ml |

The pH is 7.2 in Example 7.

Example 8 is a preferred formulation wherein the pH is about 8.5 before autoclaving and about 7.4 after autoclaving. This preferred formulation was utilized in the rising single dose study, and the rising multiple dose assessment of the safety, tolerability, and pharmacokinetics of intravenous Posaconazole in healthy human subjects, described below under the headings “Rising Single Dose Study” and “Rising Multiple Dose Study.”

### EXAMPLE 8

| Ingredient | Amount (g/1 Liter) |
|---|---|
| Posaconazole | 50 |
| Micronized, Parenteral grade | |
| 1-Palmitoyl-2-oleoyl-sn-glycerol-3- | 40 |
| phosphocholine, Powder (POPC) | |
| α,α Trehalose Dihydrate High | 250 |
| Purity (Low Endotoxin) | |
| Sodium Phosphate Monobasic | 0.040 |
| Monohydrate USP | |
| Sodium Phosphate Dibasic | 1.378 |
| Anhydrous USP | |
| Sodium Hydroxide NF | For pH adjustment |
| Phosphoric Acid NF | For pH adjustment |
| Water for Injection USP | q.s. ad 1 Liter |
| Nitrogen NF | as required |

The following is an exemplary placebo formulation wherein the pH is 6.4. This exemplary placebo formulation was utilized in the comparative stability data study described below.

| Ingredient | Concentration | |
|---|---|---|
| Placebo | | |
| POPC | 40 | mg/ml |
| Glycine | 1 | mg/ml |
| Sodium citrate dihydrate | 0.3 | mg/ml |
| Citric acid monohydrate | 0.016 | mg/ml |
| Trehalose | 250 | mg/ml |
| Water q.s. ad | 1 | ml |

### Comparative Stability Data Study

The stability of POPC in formulation Examples 1-3 was compared with the aforementioned exemplary placebo both before and after autoclaving for 20 min at 121° C. In addition, Posaconazole stability, particle size, pH, and a physical observation were ascertained for each formulation before and after autoclaving. Each formulation was also examined following an additional period of storage at 4° C., 25° C., and 40° C. (i.e., 4° C.±2° C. at 60%±5% relative humidity; 25° C.±2° C. at 60%±5% relative humidity; and 40° C.±2° C. at ambient relative humidity, respectively) for 1 month, 3 months, and 6 months after autoclaving. Notably, particle size was determined using the Malvern laser diffraction particle size analysis technique. Particle sizes are characterized by values for median (“50^{th} percentile”) and maximum (“100^{th} percentile”). The stability data from these comparative studies are compiled below for formulations reflected in Examples 1-6, shown in Tables 4-9, respectively.

TABLE 4
Stability data for Posaconazole formulation Example 1

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 51.3 | 41.4 | 1.11 | 3.77 | 7.4 | Milky white |
| Before | | | | | | | |
| Autoclaving | | | | | | | |
| Initial - | | 50.8 | 40.9 | 1.49 | 6.63 | 7.3 | Milky white |
| After | | | | | | | |
| Autoclaving | | | | | | | |
| 1 | 4° C. | 50.2 | 39.0 | 1.48 | 5.49 | 7.2 | Milky white |
| month | 25° C. | 50.7 | 39.2 | 1.48 | 5.49 | 7.2 | Milky white |
| | 40° C. | 50.6 | 39.1 | 1.49 | 5.49 | 7.1 | Milky white |
| 3 | 4° C. | 55.1 | 41.8 | 1.47 | 5.49 | 7.2 | Milky white |
| months | 25° C. | 55.4 | 41.5 | 1.48 | 6.63 | 7.2 | Milky white |
| | 40° C. | 55.4 | 40.0 | 1.49 | 6.63 | 7.1 | Milky white |
| 6 | 4° C. | 51.7 | 44.4 | 1.44 | 4.88 | 7.3 | Milky white |
| months | 25° C. | 50.3 | 42.3 | 1.50 | 5.69 | 7.3 | Milky white |
| | 40° C. | 51.5 | 36.0 | 1.57 | 5.69 | 7.3 | Milky white |

TABLE 5
Stability data for Posaconazole formulation Example 2

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 46.4 | 38.2 | 1.41 | 6.63 | 6.4 | Milky white |
| Before | | | | | | | |
| Autoclaving | | | | | | | |
| Initial - | | 46.3 | 38.2 | 1.76 | 6.63 | 6.4 | Milky white |
| After | | | | | | | |
| Autoclaving | | | | | | | |
| 1 | 4° C. | 45.8 | 37.9 | 1.70 | 6.63 | 6.4 | Milky white |
| month | 25° C. | 45.3 | 37.0 | 1.70 | 6.63 | 6.4 | Milky white |
| | 40° C. | 45.8 | 37.4 | 1.72 | 6.63 | 6.4 | Milky white |
| 3 | 4° C. | 44.8 | 36.1 | 1.69 | 6.63 | 6.4 | Milky white |
| months | 25° C. | 45.9 | 36.8 | 1.70 | 6.63 | 6.4 | Milky white |
| | 40° C. | 45.6 | 35.7 | 1.76 | 35.98 | 6.4 | Milky white |
| 6 | 4° C. | 44.1 | 38.8 | 1.65 | 6.63 | 6.6 | Milky white |
| months | 25° C. | 46.1 | 40.1 | 1.71 | 6.63 | 6.6 | Milky white |
| | 40° C. | 46.1 | 40.1 | 1.70 | 6.63 | 6.6 | Milky white |

TABLE 6
Stability data for Posaconazole formulation Example 3

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 46.1 | 36.7 | 1.39 | 5.49 | 6.6 | Milky white |
| Before | | | | | | | |
| Autoclaving | | | | | | | |
| Initial - | | 45.9 | 36.2 | 1.75 | 6.63 | 6.6 | Milky white |
| After | | | | | | | |
| Autoclaving | | | | | | | |
| 1 | 4° C. | 45.3 | 34.6 | 1.76 | 6.63 | 6.5 | Milky white |
| month | 25° C. | 44.9 | 34.4 | 1.76 | 6.63 | 6.5 | Milky white |
| | 40° C. | 44.9 | 34.5 | 1.75 | 6.63 | 6.5 | Milky white |
| 3 | 4° C. | 46.9 | 35.0 | 1.77 | 6.63 | 6.5 | Milky white |
| months | 25° C. | 46.9 | 34.9 | 1.78 | 6.63 | 6.5 | Milky white |
| | 40° C. | 47.5 | 34.5 | 1.75 | 6.63 | 6.5 | Milky white |
| 6 | 4° C. | 47.0 | 35.3 | 2.18 | 6.63 | 6.6 | Milky white |
| months | 25° C. | 46.3 | 35.1 | 1.75 | 5.69 | 6.5 | Milky white |
| | 40° C. | 49.4 | 32.5 | 2.03 | 6.63 | 6.6 | Milky white |

TABLE 7
Stability data for Posaconazole formulation Example 4

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 65.4 | 50.3 | 1.21 | 5.49 | 7.3 | Milky white |
| Before | | | | | | | |
| Autoclaving | | | | | | | |
| Initial - | | 65.5 | 50.2 | 1.66 | 6.63 | 7.2 | Milky white |
| After | | | | | | | |
| Autoclaving | | | | | | | |
| 1 | 4° C. | 65.2 | 50.2 | 1.65 | 6.63 | 7.2 | Milky white |
| month | 25° C. | 65.1 | 50.4 | 1.64 | 6.63 | 7.2 | Milky white |
| | 40° C. | 67.1 | 50.6 | 1.67 | 6.63 | 7.2 | Milky white |
| 3 | 4° C. | 68.1 | 50.9 | 1.64 | 6.63 | 7.4 | Milky white |
| months | 25° C. | 68.4 | 51.0 | 1.64 | 6.63 | 7.4 | Milky white |
| | 40° C. | 69.5 | 49.2 | 1.67 | 29.82 | 7.3 | Milky white |
| 6 | 4° C. | 66.7 | 53.3 | 1.61 | 5.68 | 7.2 | Milky white |
| months | 25° C. | 64.9 | 52.6 | 1.54 | 4.88 | 7.0 | Milky gray |
| | 40° C. | 65.4 | 47.3 | 1.64 | 56.23 | 6.8 | Milky white |

TABLE 8
Stability data for Posaconazole formulation Example 5

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 50.8 | 39.7 | 1.61 | 6.63 | 6.5 | Milky white |
| After | | | | | | | |
| Autoclaving | | | | | | | |
| 1 | 4° C. | 50.9 | 39.6 | 1.61 | 6.63 | 6.5 | Milky white |
| month | 25° C. | 51.1 | 39.8 | 1.60 | 6.63 | 6.5 | Milky white |
| | 40° C. | 50.7 | 39.4 | 1.62 | 6.63 | 6.5 | Milky white |
| 3 | 4° C. | 53.0 | 40.5 | 1.62 | 6.63 | 6.6 | Milky white |
| months | 25° C. | 53.1 | 40.6 | 1.62 | 6.63 | 6.6 | Milky white |
| | 40° C. | 51.9 | 39.0 | 1.62 | 6.63 | 6.7 | Milky white |
| 6 | 4° C. | 54.2 | 46.2 | 1.59 | 5.69 | 6.5 | Milky white |
| months | 25° C. | 53.2 | 44.6 | 1.59 | 5.69 | 6.5 | Milky white |
| | 40° C. | 52.2 | 41.2 | 1.58 | 5.69 | 6.5 | Milky white |

TABLE 9
Stability data for Posaconazole formulation Example 6

| | | | | Particle size | | | |
|---|---|---|---|---|---|---|---|
| | | | | (microns) | | | |
| Interval/ | | Posaconazole | POPC | 50^{th} | 100^{th} | | Physical |
| Condition | | (mg/ml) | (mg/ml) | percentile | percentile | pH | Observation |
| Initial - | | 46.8 | 36.5 | 1.32 | 6.63 | 6.5 | Milky white |
| Before | | | | | | | |
| Autoclaving | | | | | | | |
| Initial - | | 46.5 | 36.4 | 1.61 | 6.63 | 6.5 | Milky white |
| After | | | | | | | |
| Autoclaving | | | | | | | |
| 1 | 4° C. | 46.2 | 35.8 | 1.61 | 6.63 | 6.5 | Milky white |
| month | 25° C. | 47.6 | 36.9 | 1.60 | 6.63 | 6.5 | Milky white |
| | 40° C. | 47.3 | 36.4 | 1.62 | 6.63 | 6.5 | Milky white |
| 3 | 4° C. | 48.3 | 36.8 | 1.63 | 6.63 | 6.5 | Milky white |
| months | 25° C. | 48.6 | 37.1 | 1.62 | 6.63 | 6.6 | Milky white |
| | 40° C. | 49.1 | 36.4 | 1.61 | 6.63 | 6.4 | Milky white |
| 6 | 4° C. | 47.9 | 36.3 | 1.60 | 5.69 | 6.5 | Milky white |
| months | 25° C. | 47.3 | 36.1 | 1.60 | 5.69 | 6.5 | Milky white |
| | 40° C. | 48.8 | 34.0 | 1.60 | 5.69 | 6.5 | Milky white |

The stability of the formulation in Example 8 was determined after autoclaving for 20 min at 121° C. In addition to POPC, Posaconazole stability, particle size, pH, and a physical observation were ascertained for the formulation before and after autoclaving. The formulation was also examined following an additional period of storage at 4° C. and 25° C. (i.e., 4° C.±2° C. at 60%±5% relative humidity and 25° C.±2° C. at 60%±5% relative humidity, respectively) for 1 month, 3 months, and 6 months after autoclaving for both storage temperatures, and up to 24 months for storage at 4° C. The particle size was determined using the Sympatec laser diffraction particle size analysis technique. Particle sizes are characterized by values for median (“50^{th} percentile”) and the percentage of particles less than or equal to 10 micron (“%<10 micron”).

The stability data from this study are compiled below in Table 10 for the formulation reflected in Example 8. The formulation is stable after at least 6 months of storage at 25 deg. C., and after at least 24 months of storage at 4 deg. C.

TABLE 10
Stability data for Posaconazole formulation Example 8

| | | | | Particle size | | | |
|---|---|---|---|---|---|---|---|
| | | | | (microns) | | | |
| Interval/ | | Posaconazole | POPC | 50^{th} | % < 10 | | Physical |
| Condition | | (% LS**) | (% LS**) | percentile | micron | pH | Observation |
| Initial - | | 99.0* | 96.1 | 1.8 | 100 | 7.2 | Milky white |
| After | | | | | | | |
| Autoclaving | | | | | | | |
| 1 | 25° C. | 96.9 | 94.9 | 1.7 | 100 | 7.3 | Milky white |
| month | | | | | | | |
| 3 | 4° C. | 97.9 | 94.3 | 1.7 | 100 | 7.3 | Milky white |
| months | 25° C. | 97.4 | 92.5 | 1.7 | 100 | 7.3 | Milky white |
| 6 | 4° C. | 97.2 | 86.7 | 1.7 | 100 | 7.3 | Milky white |
| months | 25° C. | 97.3 | 83.4 | 1.8 | 100 | 7.3 | Milky white |
| 12 | 4° C. | 101.2 | 93.3 | 1.8 | 100 | 7.4 | Milky white |
| months | 25° C. | 100.6 | 86.3 | 1.9 | 100 | 7.1 | Milky white |
| 24 | 4° C. | 98.5 | 89.9 | 1.8 | 100 | 7.4 | Milky white |
| month | | | | | | | |

*average of 6 measurements;
**Percent Label Specification

The ability of the formulation in Example 8 to withstand terminal sterilization was determined after extended autoclave cycles at 12° C. Autoclave cycle times studied ranged from 20 minutes up to 150 minutes. In addition to POPC concentration, Posaconazole stability, particle size, pH, and a physical observation were ascertained for the formulation before and after extended autoclaving. The particle size was determined using the Sympatec laser diffraction particle size analysis technique. Particle sizes are characterized by values for median (“50^{th} percentile”) and the percentage of particles less than or equal to 10 microns (“%<10 micron”). Table 11 displays the characteristics of the formulation of Example 8 after extended autoclaving. These data indicate that the critical properties of the formulation including Posaconazole concentration, particle size, pH and physical observation remain relatively constant after at least 150 minutes of autoclaving at 121.5° C. In addition, the small change in POPC concentration does not affect the ability of the phospholipid to stabilize the suspension (as indicated by the particle size), and may be compensated by on overcharge of POPC during manufacture.

TABLE 11
Extended Autoclave data for Posaconazole formulation
Example 8

Particle size
(microns)

| Interval/ | Posaconazole | POPC | | % < 10 | | Physical |
|---|---|---|---|---|---|---|
| Condition | (% LS*) | (% LS*) | 50^{th} percentile | micron | pH | Observation |
| Initial - | 102.6 | 105 | 1.3 | 100 | 8.1 | Milky white |
| Before | | | | | | |
| Autoclaving | | | | | | |
| After 20 min | 102.1 | 99 | 1.7 | 100 | 7.5 | Milky white |
| Autoclaving | | | | | | |
| After 60 min | 100.3 | 96 | 1.7 | 100 | 7.4 | Milky white |
| Autoclaving | | | | | | |
| After 90 min | 100.4 | 94 | 1.7 | 100 | 7.4 | Milky white |
| Autoclaving | | | | | | |
| After 120 min | 101.7 | 91 | 1.8 | 100 | 7.3 | Milky white |
| Autoclaving | | | | | | |
| After 150 min | 101.8 | 90 | 1.8 | 100 | 7.3 | Milky white |
| Autoclaving | | | | | | |

*Percent Label Specification; each autoclave cycle at 121.5 ± 0.5° C.

The ability of the formulation in Example 8 to withstand terminal sterilization was determined after multiple autoclave cycles at 121° C. The formulation was subjected to one 20-minute autoclave cycle, followed by up to 5 additional 30-min autoclave cycles. In addition to POPC concentration, Posaconazole stability, particle size, pH, and a physical observation were ascertained for the formulation before and after extended autoclaving. The particle size was determined using the Sympatec laser diffraction particle size analysis technique. Particle sizes are characterized by values for median (“50^{th} percentile”) and the percentage of particles less than or equal to 10 micron (“%<10 micron”). Table 12 displays the characteristics of the formulation of Example 8 after extended autoclaving. These data indicate that the critical properties of the formulation including Posaconazole concentration, particle size, pH and physical observation remain relatively constant after at least five additional 30 minute cycles of autoclaving at 121.5° C. after the initial 20 minute cycle. In addition, the decrease in POPC concentration does not affect the ability of the phospholipid to stabilize the suspension (as indicated by the particle size), and may be compensated by on overcharge of POPC during manufacture

TABLE 12
Multiple Autoclave Cycle Data for Posaconazole
Formulation Example 8

| | | | Particle size | | | |
|---|---|---|---|---|---|---|
| | | | (microns) | | | |
| Interval/ | Posaconazole | POPC | 50^{th} | % < 10 | | Physical |
| Condition | (% LS) | (% LS) | percentile | micron | pH | Observation |
| Initial - | 102.6 | 105 | 1.3 | 100 | 8.1 | Milky white |
| Before | | | | | | |
| Autoclaving | | | | | | |
| After one 20 min | 102.1 | 99 | 1.7 | 100 | 7.5 | Milky white |
| cycle | | | | | | |
| After one 20 min | 102.7 | 88 | 1.8 | 100 | 7.4 | Milky white |
| cycle + one 30 min | | | | | | |
| cycle | | | | | | |
| After one 20 min | 102.5 | 89 | 1.8 | 100 | 7.3 | Milky white |
| cycle + two 30 min | | | | | | |
| cycles | | | | | | |
| After one 20 min | 104.2 | 90 | 1.8 | 100 | 7.3 | Milky white |
| cycle + three 30 min | | | | | | |
| cycles | | | | | | |
| After one 20 min | 102.2 | 87 | 1.9 | 100 | 7.2 | Milky white |
| cycle + four 30 min | | | | | | |
| cycles | | | | | | |
| After one 20 min | 102.3 | 85 | 1.9 | 100 | 7.2 | Milky white |
| cycle + five 30 min | | | | | | |
| cycles | | | | | | |

The data in Tables 10-12 reflect the robust stability of the behavior of the particle size distributions that results from the inventors' formulations, and in particular, of the formulation of Example 8. These data reflect the storage stability, as well as the formulations' resistance to potentially deleterious effects of various autoclaving cycles.

### In Vitro Activity Data

Activities of Posaconazole against a broad spectrum of infectious agents have been tested in vitro. Tables 13 and 14 display a subset of the results of this in vitro testing, showing some of those infectious agents against which Posaconazole is most active. It is believed that Posaconazole will also show activity against other infectious agents not listed in Tables 13 and 14, such as those causing phaeohyphomycosis.

TABLE 13
Geometric Mean MIC and MIC[90] Values (μg/mL) for Strains Tested (n) in Posaconazole (POS),
Fluconazole (FLU) and Itraconazole (ITZ)

| | POS | | | FLZ | | | ITZ | | |
|---|---|---|---|---|---|---|---|---|---|
| Organism | n | Mean | MIC[90] | n | Mean | MIC[90] | n | Mean | MIC[90] |
| Aspergillus flavus | 241 | 0.079 | 0.25 | 94 | 220.898 | 256.0 | 203 | 0.213 | 1.0 |
| Aspergillus fumigatus | 2,158 | 0.118 | 0.5 | 735 | 247.922 | 512.0 | 1560 | 0.397 | 1.0 |
| Aspergillus nidulans | 33 | 0.055 | 0.25 | 8 | 76.109 | (32.0-128.0) | 21 | 0.186 | 0.5 |
| Aspergillus niger | 171 | 0.195 | 0.5 | 64 | 234.753 | 256.0 | 153 | 0.834 | 2.0 |
| Aspergillus sydowii | 8 | 0.177 | (0.031-0.5) | 7 | 115.933 | (64.0-256.0) | 8 | 0.500 | (0.125-2.0) |
| Aspergillus terreus | 100 | 0.052 | 0.25 | 37 | 208.327 | 256.0 | 56 | 0.229 | 0.5 |
| Aspergillus ustus | 7 | 1.641 | (0.25-8.0) | 7 | 172.275 | (64.0-256.0) | 7 | 0.906 | (0.125-2.0) |
| Candida albicans | 8,847 | 0.037 | 0.25 | 7,879 | 0.415 | 2.0 | 7,686 | 0.064 | 0.25 |
| Candida dubliniensis | 339 | 0.062 | 0.25 | 231 | 0.454 | 32.0 | 197 | 0.107 | 0.5 |
| Candida glabrata | 2,507 | 0.672 | 2.0 | 2,197 | 9.719 | 64.0 | 2,188 | 0.853 | 4.0 |
| Candida krusei | 496 | 0.335 | 1.0 | 386 | 32.521 | 64.0 | 383 | 0.576 | 1.0 |
| Candida parapsilosis | 2,126 | 0.073 | 0.125 | 1,916 | 0.910 | 2.0 | 1,903 | 0.161 | 0.5 |
| Cryptococcus laurentii | 5 | 0.095 | (0.008-0.5) | 3 | 5.040 | (4.0-8.0) | 3 | 0.397 | (0.25-0.5) |
| Cryptococcus neoformans | 1,427 | 0.119 | 0.25 | 1,237 | 1.781 | 8.0 | 1,269 | 0.444 | 4.0 |
| Coccidioides immitis | 50 | 0.304 | 1.0 | 25 | 16.450 | 32.0 | 50 | 0.198 | 0.25 |
| Fonsecae pedrosoi | 4 | 0.250 | (0.25) | 2 | 64.000 | (64.0) | 4 | 0.063 | (0.008-0.5) |
| Histoplasma capsulatum | 58 | 0.038 | 0.25 | 8 | 19.027 | (8.0-32.0) | 53 | 0.018 | 0.063 |
| Pseudallescheria boydii | 66 | 0.365 | 1.0 | 41 | 41.237 | 128.0 | 61 | 0.506 | 1.0 |
| Alternaria spp | 13 | 0.101 | 0.25 | 0 | — | — | 13 | 0.326 | 1.0 |
| Exophiala dermatidis | 3 | 0.125 | (0.125) | 2 | 8.000 | (8.0) | 2 | 1.000 | (1.0) |
| Exophiala jeanselmei | 10 | 0.287 | 0.5 | 0 | — | — | 10 | 0.467 | 1.0 |
| Exophiala moniliae | 2 | 0.016 | (0.016) | 0 | — | — | 2 | 0.031 | (0.031) |
| Fusarium spp | 38 | 2.319 | 16.0 | 27 | 249.512 | 256.0 | 30 | 13.300 | 16.0 |
| Ramichloridium obovoideum | 2 | 0.044 | (0.031-0.063) | 2 | 22.627 | (16.0-32.0) | 2 | 0.016 | (0.016) |
| Rhizomucor spp | 2 | 0.016 | (0.016) | 0 | — | — | 2 | 0.016 | (0.016) |
| Mucor spp | 17 | 0.694 | 16.0 | 10 | 207.937 | 256.0 | 12 | 2.378 | 16.0 |
| Rhizopus spp | 29 | 1.000 | 4.0 | 19 | 229.461 | 256.0 | 21 | 3.281 | 16.0 |
| Candida famata | 44 | 0.125 | 0.5 | 44 | 4.084 | 32.0 | 27 | 0.348 | 1.0 |
| Candida guilliermondii | 143 | 0.178 | 0.5 | 106 | 4.000 | 32.0 | 82 | 0.479 | 1.0 |
| Candida lusitaniae | 306 | 0.048 | 0.125 | 221 | 0.627 | 2.0 | 202 | 0.216 | 1.0 |
| Candida kefyr | 53 | 0.081 | 0.25 | 51 | 0.500 | 4.0 | 39 | 0.188 | 0.5 |
| Candida rugosa | 26 | 0.039 | 0.5 | 21 | 3.391 | 16.0 | 17 | 0.196 | 4.0 |
| Candida tropicalis | 1,645 | 0.081 | 0.25 | 1,476 | 0.961 | 4.0 | 1,450 | 0.167 | 0.5 |
| Candida-zeylanoides | 4 | 0.031 | (0.008-0.25) | 4 | 0.354 | (0.125-1.0) | 4 | 0.105 | (0.031-0.5) |
| Kluyveromyces marxianus | 6 | 0.079 | (0.063-0.25) | 6 | 0.500 | (0.25-1.0) | 6 | 0.070 | (0.031-0.125) |
| Saccharomyces cerevisiae | 86 | 0.249 | 1.0 | 59 | 2.845 | 16.0 | 54 | 0.418 | 2.0 |
| Yarrowia lipolytica | 5 | 0.144 | (0.016-1.0) | 5 | 1.741 | (0.125-32.0) | 0 | — | — |
| Pichia anomala | 13 | 0.689 | 1.0 | 12 | 2.670 | 4.0 | 12 | 0.375 | 1.0 |
| Pichia etchel | 2 | 0.125 | (0.125) | 2 | 0.125 | (0.125) | 0 | — | — |
| Pichia ohmeri | 1 | 0.016 | (0.016) | 1 | 4.000 | (4.0) | 0 | — | — |
| Trichosporon spp | 6 | 0.630 | (0.5-1.0) | 6 | 12.699 | (4.0-64.0) | 6 | 1.123 | (0.5-2.0) |
| Bjerkandera adusta | 14 | 0.250 | 0.25 | 14 | 4.000 | 4.0 | 14 | 0.057 | 0.063 |
| Blastomyces dermatitidis | 43 | 0.053 | 0.125 | 38 | 2.191 | 16.0 | 38 | 0.045 | 2.0 |
| Epidermophyton floccosum | 70 | 0.029 | 0.125 | 15 | 1.447 | 2.0 | 18 | 0.088 | 32.0 |
| Paracoccidioides brasiliensis | 13 | 0.048 | 0.125 | 13 | 0.766 | 4.0 | 13 | 0.025 | 0.063 |
| Scedosporium apiospermum | 32 | 0.173 | 1.0 | 15 | 84.449 | 256.0 | 26 | 1.341 | 32.0 |
| Sporothrix schenckii | 16 | 0.771 | 2.0 | 0 | — | — | 11 | 0.302 | 0.5 |
| Wangiella dermatitidis | 4 | 0.088 | (0.063-0.125) | 2 | 256.000 | (256.0) | 4 | 0.500 | (0.063-1.0) |
| Absidia spp | 8 | 0.177 | (0.031-0.5) | 6 | 143.675 | (128.0-256.0) | 8 | 0.229 | (0.063-2.0) |
| Apophysomyces spp | 9 | 0.340 | (0.031-4.0) | 4 | 128.000 | (128.0) | 6 | 0.707 | (0.031-8.0) |
| Bipolaris spp | 8 | 0.354 | (0.125-1.0) | 2 | 64.000 | (64.0) | 2 | 0.250 | (0.25) |
| Curvularia spp | 5 | 0.072 | (0.031-0.125) | 2 | 16.000 | (16.0) | 2 | 0.500 | (0.5) |
| Microsporum audouinii | 1 | 0.250 | (0.25) | 0 | — | — | 1 | 0.125 | (0.125) |
| Microsporum canis | 86 | 0.034 | 0.5 | 11 | 2.000 | 4.0 | 23 | 0.041 | 2.0 |
| Microsporum fulvum | 1 | 0.500 | (0.5) | 0 | — | — | 1 | 4.000 | (4.0) |
| Microsporum gypseum | 5 | 0.042 | (0.008-0.5) | 4 | 16.000 | (4.0-128.0) | 4 | 0.044 | (0.016-0.5) |
| Microsporum persicolor | 1 | 0.250 | (0.25) | 1 | 128.000 | (128.0) | 1 | 0.500 | (0.5) |
| Paecilomyces spp | 16 | 0.239 | 0.5 | 14 | 141.323 | 256.0 | 14 | 0.640 | 2.0 |
| Penicillium spp | 93 | 0.308 | 1.0 | 66 | 220.996 | 256.0 | 83 | 0.853 | 2.0 |
| Trichophyton mentagrophytes | 84 | 0.036 | 0.125 | 30 | 9.190 | 64.0 | 29 | 0.041 | 0.25 |
| Trichophyton raubitschekii | 1 | 0.250 | (0.25) | 1 | 128.000 | (128.0) | 1 | 0.250 | (0.25) |
| Trichophyton rubrum | 148 | 0.047 | 0.25 | 93 | 4.407 | 32.0 | 91 | 0.074 | 0.25 |
| Trichophyton soudanense | 1 | 0.500 | (0.5) | 1 | 128.000 | (128.0) | 1 | 4.000 | (4.0) |
| Trichophyton spp | 10 | 0.036 | 0.064 | 0 | — | — | 0 | — | — |
| Trichophyton terrestre | 1 | 0.125 | (0.125) | 1 | 1.000 | (1.0) | 1 | 0.250 | (0.25) |
| Trichophyton tonsurans | 74 | 0.029 | 0.125 | 24 | 3.775 | 32.0 | 23 | 0.036 | 0.063 |

TABLE 14
Geometric Mean MIC and MIC[90] Values (μg/mL) for # Strains Tested (n) in Posaconazole (POS),
Amphotericin (AMB) and Voriconazole (VOR)

| | POS | | | AMB | | | VOR | | |
|---|---|---|---|---|---|---|---|---|---|
| Organism | n | Mean | MIC[90] | n | Mean | MIC[90] | n | Mean | MIC[90] |
| Aspergillus flavus | 241 | 0.079 | 0.25 | 177 | 0.910 | 2.0 | 89 | 0.339 | 1.0 |
| Aspergillus fumigatus | 2,158 | 0.118 | 0.5 | 1,567 | 0.683 | 1.0 | 1149 | 0.282 | 0.5 |
| Aspergillus nidulans | 33 | 0.055 | 0.25 | 20 | 0.758 | 2.0 | 6 | 0.070 | (0.031-0.125) |
| Aspergillus niger | 171 | 0.195 | 0.5 | 152 | 0.360 | 1.0 | 101 | 0.480 | 2.0 |
| Aspergillus sydowii | 8 | 0.177 | (0.031-0.5) | 3 | 1.260 | (1.0-2.0) | 3 | 0.397 | (0.25-1.0) |
| Aspergillus terreus | 100 | 0.052 | 0.25 | 54 | 1.759 | 4.0 | 22 | 0.312 | 0.5 |
| Aspergillus ustus | 7 | 1.641 | (0.25-8.0) | 7 | 0.673 | (0.25-1.0) | 4 | 1.189 | (0.25-2.0) |
| Candida albicans | 8,847 | 0.037 | 0.25 | 6,651 | 0.686 | 1.0 | 3,790 | 0.021 | 0.063 |
| Candida dubliniensis | 339 | 0.062 | 0.25 | 211 | 0.513 | 1.0 | 177 | 0.028 | 0.125 |
| Candida glabrata | 2,507 | 0.672 | 2.0 | 1,881 | 0.798 | 1.0 | 1,264 | 0.305 | 2.0 |
| Candida krusei | 496 | 0.335 | 1.0 | 282 | 0.976 | 2.0 | 210 | 0.346 | 0.5 |
| Candida parapsilosis | 2,126 | 0.073 | 0.125 | 1,655 | 0.761 | 1.0 | 1,011 | 0.036 | 0.125 |
| Cryptococcus laurentii | 5 | 0.095 | (0.008-0.5) | 3 | 0.794 | (0.5-1.0) | 1 | 0.250 | (0.25) |
| Cryptococcus neoformans | 1,427 | 0.119 | 0.25 | 1,122 | 0.667 | 1.0 | 277 | 0.054 | 0.125 |
| Coccidioides immitis | 50 | 0.304 | 1.0 | 25 | 0.390 | 0.5 | 0 | — | — |
| Fonsecae pedrosoi | 4 | 0.250 | (0.25) | 2 | 1.000 | (1.0) | 1 | 0.500 | (0.5) |
| Histoplasma capsulatum | 58 | 0.038 | 0.25 | 53 | 0.250 | 0.5 | 0 | — | — |
| Pseudallescheria boydii | 66 | 0.365 | 1.0 | 41 | 1.718 | 4.0 | 3 | 0.250 | (0.125-0.5) |
| Alternaria spp | 13 | 0.101 | 0.25 | 13 | 0.852 | 4.0 | 0 | — | — |
| Exophiala dermatidis | 3 | 0.125 | (0.125) | 2 | 0.500 | (0.5) | 1 | 0.063 | (0.063) |
| Exophiala jeanselmei | 10 | 0.287 | 0.5 | 10 | 0.660 | 1.0 | 9 | 0.794 | (0.5-1.0) |
| Exophiala moniliae | 2 | 0.016 | (0.016) | 2 | 0.177 | (0.125-0.25) | 0 | — | — |
| Fusarium spp | 38 | 2.319 | 16.0 | 30 | 1.203 | 2.0 | 14 | 4.416 | 16.0 |
| Ramichloridium obovoideum | 2 | 0.044 | (0.031-0.063) | 2 | 1.000 | (1.0) | 0 | — | — |
| Rhizomucor spp | 2 | 0.016 | (0.016) | 2 | 0.063 | (0.063) | 1 | 2.000 | (2.0) |
| Mucor spp | 17 | 0.694 | 16.0 | 15 | 0.274 | 1.0 | 8 | 24.675 | (1.0-128.0) |
| Rhizopus spp | 29 | 1.000 | 4.0 | 29 | 0.635 | 2.0 | 9 | 8.000 | (1.0-32.0) |
| Candida famata | 44 | 0.125 | 0.5 | 28 | 0.841 | 2.0 | 5 | 0.072 | (0.008-0.5) |
| Candida guilliermondii | 143 | 0.178 | 0.5 | 76 | 0.553 | 1.0 | 26 | 0.112 | 8.0 |
| Candida lusitaniae | 306 | 0.048 | 0.125 | 164 | 0.506 | 1.0 | 89 | 0.023 | 0.063 |
| Candida kefyr | 53 | 0.081 | 0.25 | 25 | 0.779 | 1.0 | 14 | 0.031 | 0.031 |
| Candida rugosa | 26 | 0.039 | 0.5 | 17 | 0.665 | 1.0 | 15 | 0.072 | 0.5 |
| Candida tropicalis | 1,645 | 0.081 | 0.25 | 1,209 | 0.774 | 1.0 | 765 | 0.075 | 0.5 |
| Candida zeylanoides | 4 | 0.031 | (0.008-0.25) | 3 | 1.000 | (1.0) | 3 | 0.039 | (0.008-0.125) |
| Kluyveromyces marxianus | 6 | 0.079 | (0.063-0.25) | 6 | 1.000 | (1.0) | 0 | — | — |
| Saccharomyces cerevisiae | 86 | 0.249 | 1.0 | 38 | 0.775 | 1.0 | 22 | 0.050 | 0.125 |
| Yarrowia lipolytica | 5 | 0.144 | (0.016-1.0) | 2 | 0.500 | (0.5) | 0 | — | — |
| Pichia anomala | 13 | 0.689 | 1.0 | 10 | 0.707 | 1.0 | 0 | — | — |
| Pichia etchel | 2 | 0.125 | (0.125) | 2 | 0.063 | (0.063) | 0 | — | — |
| Pichia ohmeri | 1 | 0.016 | (0.016) | 0 | — | — | 0 | — | — |
| Trichosporon spp | 6 | 0.630 | (0.5-1.0) | 5 | 1.320 | (1.0-2.0) | 1 | 0.125 | (0.125) |
| Bjerkandera adusta | 14 | 0.250 | 0.25 | 14 | 0.215 | 0.25 | 14 | 0.216 | 0.25 |
| Blastomyces dermatitidis | 43 | 0.053 | 0.125 | 38 | 0.153 | 0.5 | 0 | — | — |
| Epidermophyton floccosum | 70 | 0.029 | 0.125 | 0 | — | — | 10 | 0.015 | 0.016 |
| Paracoccidioides brasiliensis | 13 | 0.048 | 0.125 | 13 | 0.096 | 0.25 | 0 | — | — |
| Scedosporium apiospermum | 32 | 0.173 | 1.0 | 27 | 2.274 | 8.0 | 14 | 0.098 | 0.5 |
| Sporothrix schenckii | 16 | 0.771 | 2.0 | 10 | 0.574 | 1.0 | 0 | — | — |
| Wangiella dermatitidis | 4 | 0.088 | (0.063-0.125) | 3 | 0.630 | (0.25-1.0) | 1 | 1.000 | (1.0) |
| Absidia spp | 8 | 0.177 | (0.031-0.5) | 8 | 0.545 | (0.25-2.0) | 6 | 40.318 | (8.0-128.0) |
| Apophysomyces spp | 9 | 0.340 | (0.031-4.0) | 7 | 0.500 | (0.031-4.0) | 5 | 42.224 | (16.0-128.0) |
| Bipolaris spp | 8 | 0.354 | (0.125-1.0) | 2 | 0.250 | (0.25) | 1 | 1.000 | (1.0) |
| Curvularia spp | 5 | 0.072 | (0.031-0.125) | 2 | 0.500 | (0.5) | 1 | 0.125 | (0.125) |
| Microsporum audouinii | 1 | 0.250 | (0.25) | 0 | — | — | 0 | — | — |
| Microsporum canis | 86 | 0.034 | 0.5 | 0 | — | — | 10 | 0.018 | 0.031 |
| Microsporum fulvum | 1 | 0.500 | (0.5) | 0 | — | — | 0 | — | — |
| Microsporum gypseum | 5 | 0.042 | (0.008-0.5) | 0 | — | — | 0 | — | — |
| Microsporum persicolor | 1 | 0.250 | (0.25) | 0 | — | — | 0 | — | — |
| Paecilomyces spp | 16 | 0.239 | 0.5 | 14 | 1.104 | 16.0 | 8 | 0.324 | (0.031-2.0) |
| Penicillium spp | 93 | 0.308 | 1.0 | 83 | 1.025 | 4.0 | 35 | 0.622 | 2.0 |
| Trichophyton mentagrophytes | 84 | 0.036 | 0.125 | 2 | 1.000 | (1.0) | 11 | 0.038 | 0.25 |
| Trichophyton raubitschekii | 1 | 0.250 | (0.25) | 0 | — | — | 0 | — | — |
| Trichophyton rubrum | 148 | 0.047 | 0.25 | 0 | — | — | 10 | 0.021 | 0.063 |
| Trichophyton soudanense | 1 | 0.500 | (0.5) | 0 | — | — | 0 | — | — |
| Trichophyton spp | 10 | 0.036 | 0.064 | 0 | — | — | 0 | — | — |
| Trichophyton terrestre | 1 | 0.125 | (0.125) | 0 | — | — | 0 | — | — |
| Trichophyton tonsurans | 74 | 0.029 | 0.125 | 3 | 1.260 | (0.5-8.0) | 11 | 0.043 | 0.063 |

### Rising Single Dose Study

The pharmacokinetic characteristics of a stable Posaconazole intravenous suspension formulation according to the present invention were evaluated in a Phase-1, single-site, randomized, evaluator-blinded (within dose level), placebo-controlled, rising-single-dose study, with up to six groups of 12 healthy subjects. The purpose of the study was to evaluate the safety, tolerability, and pharmacokinetics of the Posaconazole intravenous drug product formulation (hereinafter referred to as “POS IV”) when delivered intravenously. Table 15 shows the POS IV formulation, and Table 16 shows the physical characteristics of this formulation after sterilization, but before dilution in 5% dextrose.

TABLE 15
POS IV Formulation, 50 mg/mL

| Ingredient | mg/mL |
|---|---|
| Posaconazole, micronized | 50.0 |
| 1-Palmitoyl-2-Oleoyl-sn-glycero-3-Phosphocholine, | 40.0 |
| Powder, Endotoxin tested (POPC) | |
| Sodium Phoshate, Monobasic, Monohydrate, Crystal, USP | 0.040 |
| Sodium Phosphate, Dibasic, Anhydrous, USP | 1.378 |
| Trehalose Dihydrate High Purity (Low Endotoxin) | 250.0 |
| Sodium Hydroxide, N.F. (1.0 N) | AN |
| Phosphoric Acid, N.F. (20% w/w) | AN |
| Water Injection, USP, q.s. ad | 1 |

AN = As needed for pH adjustment

TABLE 16
Physical Characteristics of POS IV, 50 mg/mL

| | |
|---|---|
| Description: | milky liquid |
| pH: | ~6-8 |
| Osmolality: | ~875 mOsm/kg (isotonic upon |
| | admixing) |
| Particulates: | Less than 3000 particles of 10 |
| | microns or larger per small volume |
| | parenteral unit; |
| | Less than 300 particles of 25 |
| | microns or larger per small volume |
| | parenteral unit |
| Particle Size: | Median 1.8 μm, 100% ≦10 μm |
| Sterility/ | meets USP tests |
| Endotoxins: | |

Within each dose group, subjects were randomized on Day 1 according to a computer-generated schedule provided by Schering-Plough Research Institute.

Healthy adult males or females 18 to 45 years of age having body mass indices (BMIs) of 19 to 27 were eligible for inclusion in Groups 1 to 4 of the study. Healthy adult males or females 18 to 45 years of age having BMIs of 19 to 27 and having body weights of greater than 60 kg were eligible for inclusion in Groups 5 and 6 of the study.

POS IV (50 mg/mL) was diluted in 5% dextrose in water (D₅W) in IV bags. Subjects assigned to active drug received in a 100-mL volume one of the following single doses administered intravenously over 1 hour: Group 1, mg; Group 2, 50 mg; Group 3, 100 mg; Group 4, 200 mg; Group 5, 400 mg; Group 6, a 125-mL volume a single dose of 600 mg administered intravenously over 1 hour and 15 minutes.

Blood samples (10 mL each) for the determination of Posaconazole concentrations were collected immediately prior to dosing (0 hour), and at 0.25, 0.5, 1, 1.25, 1.5, 2, 3, 4, 6, 8, 10, 12, 14, 16, 24, 48, 72, 96, and 120 hours after the start of infusion, as well as on the follow-up visit on Day 14. The blood samples were collected into two tubes containing ethylenediaminetetraacetate salt (EDTA) with each tube containing 4 mL to 5 mL of blood, one tube for determination of Posaconazole in whole blood and other in plasma. For determination of Posaconazole in plasma, the tube of blood (4 mL to 5 mL) was centrifuged within approximately 15 minutes of collection at approximately 4° C. and 1500 g for 10 minutes to completely separate red blood cells from plasma. All blood and plasma samples were immediately frozen to at least −20° C. and maintained in the frozen state until assayed. The blood and plasma concentrations of Posaconazole were determined using validated high performance liquid chromatographic-mass spectrometric (LC-MS/MS) assays. The lower limit of quantitation (LLOQ) of this assay was 5.0 ng/mL and the calibration range was 5 to 5000 ng/mL.

The following pharmacokinetic parameters were determined: maximum plasma concentration (Cₘₐₓ); time of maximum plasma concentration (Tₘₐₓ); the area under the plasma concentration versus time curve to infinity (AUC[I]); the area under the plasma concentration versus time curve to the final measurable sampling time (AUC[tf]); terminal phase half-life (t_{1/2}); total body clearance (CL); and, volume of distribution at steady-state (Vdss).

Posaconazole blood and plasma concentrations above the LLOQ were used for the non-compartmental pharmacokinetic analyses. Pharsight® Knowledgebase Server®: version 2.0.1 (PKS) with WinNonlin version 4.0.1 (Pharsight Corporation, Cary, N.C.) was used to conduct the pharmacokinetic analysis. The Cₘₐₓ and Tₘₐₓ were the observed values. The terminal phase rate constant (k) was calculated as the negative of the slope of the log-linear terminal portion of the serum concentration-time curve using linear regression. The terminal phase half-life, t_{1/2}, was calculated as 0.693/k.

The area under the serum concentration-time curve from time 0 to the time of final quantifiable sample [AUC(tf)] was calculated using the linear trapezoidal rule. AUC(tf) was then extrapolated to infinity (I) as follows: AUC(I)=AUC(tf)+Ces(tf)/k where Ces(tf) is the estimated concentration determined from linear regression at final measurable sampling time, tf. Total body clearance, CL, was calculated by the following equation:

CL=Dose/AUC(I)

The apparent volume of distribution at steady-state, Vdss, was calculated as:

Vdss=CL×MRT

where MRT is the mean residence time (adjusted for infusion duration) determined from moment analysis.

The observed single dose plasma concentrations were used for pharmacokinetic modeling and simulation and to project steady-state concentrations for once-a-day (QD) dosing regimen. A nonparameteric superposition method was used for the pharmacokinetic modeling and simulation under the assumption of linear pharmacokinetics (see Gibaldi M, Perrier D., Pharmacokinetics, 2nd ed., New York: Marcel Dekker, Inc., 1982:409-17).

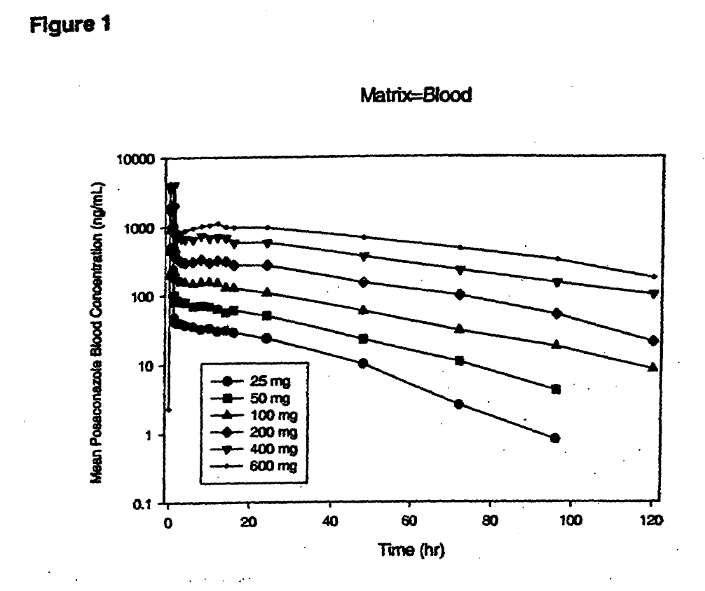

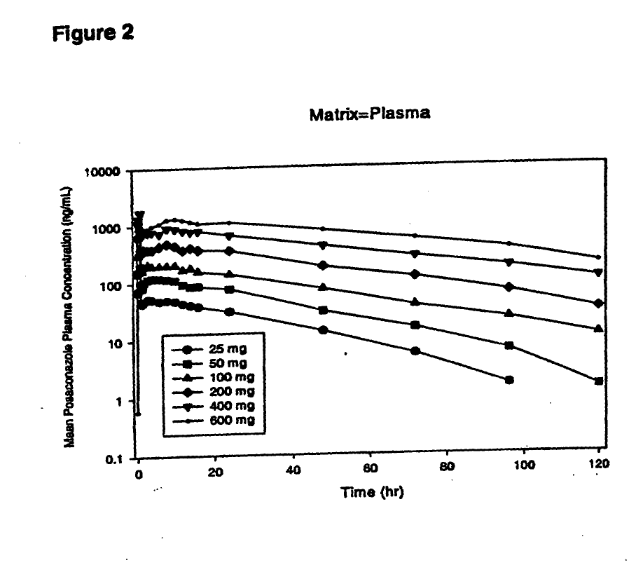

After cessation of infusion of POS IV, Posaconazole plasma concentrations declined unusually rapidly, and then, surprisingly, increased subsequently, followed by a slow declining terminal phase (see FIGS. 1-4). This pharmacokinetic profile is believed to be atypical and unique among known azoles. Moreover, this pharmacokinetic pattern was also observed after the intravenous administration of Posaconazole in animals. It is indicative of a rapid distribution of Posaconazole to the liver and spleen and subsequent slow release from these tissues. Therefore, as noted in the literature with respect to another pharmaceutically active agent (Townsend R W, Zutshi A, Bekersky I., “Biodistribution of 4-[14C]cholesterol-Ambisome following a single intravenous administration to rats”, Drug Metabolism and Disposition. 2001; 29: 681-5 (2001), POS IV may be initially sequestrated in tissues, such as the liver and spleen, via uptake through the reticuloendothelial system (“RES”). Although not intended to be limited to any single mechanism of action, it is believed that the resulting high concentrations of Posaconazole in these tissues due to sequestration of the drug may contribute to enhanced anti-infective activity, since these tissues are often the sites of infection.

In order to determine the target dosing for intravenous administration, it was necessary to determine a target range for mean C_{avg} and mean Cₘₐₓ. Previous studies on orally administered Posaconazole are instructive in this regard. Table 17 displays pharmacokinetic data resulting from such oral administration, arranged by quartile based on the observed range of Posaconazole plasma concentration values. For each quartile, the response rate for aspergillosis is displayed.

TABLE 17
Pharmacokinetic Results of Orally Administered
Posaconazole

| | Plasma Cₘₐₓ | | Plasma C_{avg} | | |
|---|---|---|---|---|---|
| | (ng/mL) | | (ng/mL) (b) | | Response |
| Quartile | Mean (a) | % CV | Mean (a) | % CV | (%) |
| 1 | 142 | 51 | 134 | 45 | 24 |
| 2 | 467 | 27 | 411 | 21 | 53 |
| 3 | 852 | 15 | 719 | 12 | 53 |
| 4 | 1480 | 16 | 1250 | 28 | 71 |

(a): n = 17, with the exception of Quartile 4 where n = 16
(b): C_{avg} = average plasma concentration across time points in the same subject; an estimate of AUC is obtained by C_{avg} by the dosing interval.

The table shows that the target mean Cₘₐₓ for a response rate of at least 50% should be in the range of 467 to 1480 ng/mL, or higher. The pharmacokinetic modeling and steady-state projection based on the pharmacokinetic results of POS IV once-a-day (QD) dosing regimen show that the projected Posaconazole mean Cₘₐₓ at a 100 mg POS IV QD dose will be 714 ng/mL (see Table 20, 100 mg dose), which exceeds 467 ng/mL, the minimum clinically relevant mean plasma Cₘₐₓ. The data in Table 20 suggest that there exists a dose between 50 and 100 mg which will result in the minimum clinically relevant mean plasma Cₘₐₓ of about 467. However, in treating a patient with a serious fungal infection, it is desirable to treat the patient with the maximum tolerated dose. Thus, having established that a dose of 100 mg is projected to achieve the minimum clinically relevant mean plasma Cₘₐₓ, it may be desirable to dose at higher quantities, e.g., 200 mg, 400 mg, or 600 mg, subject to tolerability.

At the same time, posaconazole has a long half-life and a large volume of distribution that results in sustained plasma levels and extensive penetration into body tissues with a likely accumulation in tissues. As a result, the efficacy against fungal diseases has been demonstrated in patients with mean Cₘₐₓ as low as 142 ng/mL with 800 mg/day oral suspension as a divided dose (see Table 17). Thus, it can be inferred that posaconazole's long half-life and large volume of distribution results in sustained plasma and tissue levels allowing for safer and better tolerated pharmacokinetic behavior.

After intravenous administration of POS IV formulation, Posaconazole was slowly eliminated from plasma with an average terminal half-life of 21 to 39 hours. The half-life was higher at the higher dose compared to that at lower dose groups (see Tables 17 and 18), in a range of about 15 hours (with a 100 mg dose) to about 51 hours (with a 400 mg dose). In certain instances, a long half-life is desirable as it provides the sustained and high plasma concentration of antifungal agent over the entire dosing interval, likely contributing to better antifungal activity. The systemic clearance ranged from 6 to 13 L/hr (see Tables 18 and 19). The mean volume of distribution was large (326 to 408 L) exceeding total body water volume of about 40 L. This suggests extensive tissue distribution and penetration into the tissues, a characteristic that likely contributes to enhanced anti-infective activity. The range in the data for Vdss was from 219 to 516 L. This is consistent with the coefficient of variation of the data, which suggests that the volume distribution could have a range of 200 to 500 L.

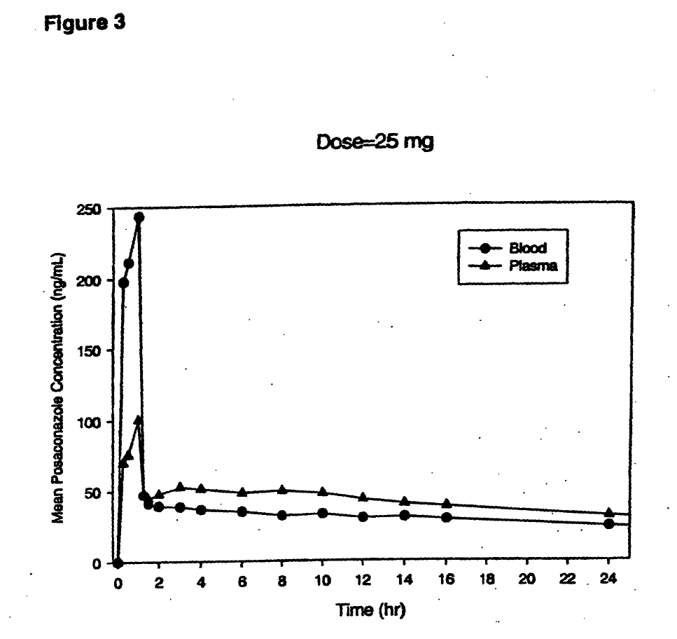

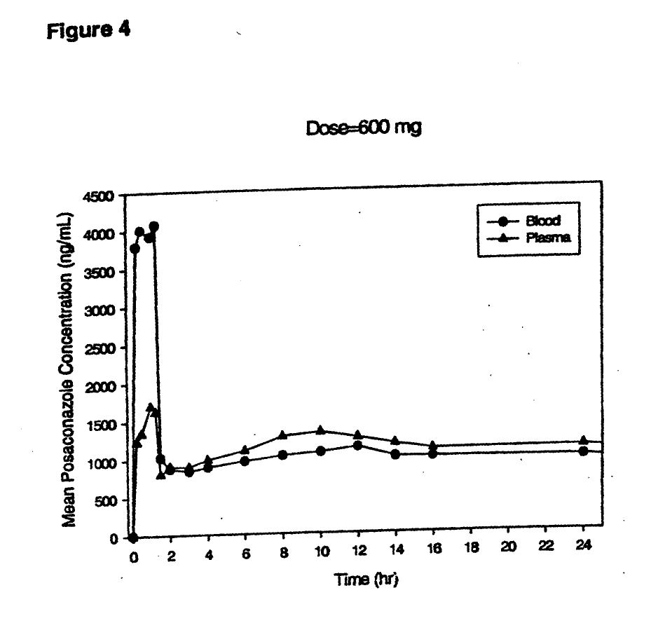

The preferable ratios of blood to plasma Posaconazole Cₘₐₓ and AUC values are shown in Tables 21 and 22. Overall Posaconazole exposure (AUC) was higher in plasma compared to that in blood (see Tables 21 and 22—AUC ratio). However, the Posaconazole concentrations were greater in blood than in plasma during the infusion and approximately up to 1 hr post-infusion (see FIGS. 3 and 4; Tables 21 and 22, Cₘₐₓ ratio). These unique differences between blood and plasma concentrations may contribute to the preferential sequestration of Posaconazole in the liver and spleen, as previously noted. The coefficient of variation of the data suggests that the ratio of blood to plasma Posaconazole Cₘₐₓ could have a range of 1.8 to 3.5 for single dose infused over 1 hour to deliver 25-600 mg of Posaconazole. The coefficient of variation of the data suggests that the ratio of blood to plasma Posaconazole Cₘₐₓ could have a range of 1.0 to 2.3 at steady state when Posaconazole is infused over about 1 hour, and repeated on a 24-hour basis, to deliver 25-600 mg of Posaconazole. A ratio different than that shown in Table 22 may provide different distribution properties that could translate into differences in anti-infective activity.

TABLE 18
Mean (n = 9) Posaconazole Blood Pharmacokinetic Parameters in Subjects after a Single-dose
1 hr IV Infusion of Posaconazole IV Formulation

| | 25 mg | | 50 mg | | 100 mg | | 200 mg | | 400 mg | | 600 mg | |
|---|---|---|---|---|---|---|---|---|---|---|---|---|
| Parameter | Mean | CV (%) | Mean | CV (%) | Mean | CV (%) | Mean | CV (%) | Mean | CV (%) | Mean | CV (%) |
| Cmax | 244 | 18 | 540 | 11 | 1130 | 22 | 2150 | 8 | 4330 | 14 | 4410 | 9 |
| (ng/mL) | | | | | | | | | | | | |
| Tmax^{a} (hr) | 1 | — | 1 | — | 1 | — | 1 | — | 1 | — | 1.25 | — |
| range | 0.5-1.0 | | 0.5-1.0 | | 0.5-1.0 | | 0.5-1.0 | | 0.25-1.0 | | 0.5-1.25 | |
| AUC (tf) | 1450 | 28 | 3370 | 16 | 8110 | 21 | 19500 | 20 | 44800 | 23 | 76300 | 15 |
| (ng · hr/mL) | | | | | | | | | | | | |
| AUC(I) | 1680 | 27 | 3620 | 17 | 8630 | 22 | 20700 | 21 | 50900 | 29 | 87300 | 16 |
| (ng · hr/mL) | | | | | | | | | | | | |
| t½ (hr) | 21.1 | 25 | 22.6 | 19 | 27.8 | 23 | 26.5 | 19 | 37.5 | 31 | 39.0 | 22 |
| CL | 15.7 | 23 | 14.2 | 18 | 12.3 | 31 | 10.1 | 24 | 8.33 | 23 | 7.01 | 14 |
| (L/hr) | | | | | | | | | | | | |
| Vdss (L) | 408 | 14 | 395 | 11 | 408 | 12 | 379 | 19 | 427 | 22 | 393 | 24 |

^{a}median

TABLE 19
Mean (n = 9) Posaconazole Plasma Pharmacokinetic Parameters in Subjects after a Single-dose
1 hr IV Infusion of Posaconazole IV Formulation

| | 25 mg | | 50 mg | | 100 mg | | 200 mg | | 400 mg | | 600 mg | |
|---|---|---|---|---|---|---|---|---|---|---|---|---|
| Parameter | Mean | CV (%) | Mean | CV (%) | Mean | CV (%) | Mean | CV (%) | Mean | CV (%) | Mean | CV (%) |
| Cmax | 103 | 25 | 206 | 26 | 426 | 25 | 898 | 27 | 1780 | 21 | 1850 | 21 |
| (ng/mL) | | | | | | | | | | | | |
| Tmax^{a} (hr) | 1 | — | 1 | — | 1 | — | 1 | — | 1 | — | 1.25 | — |
| range | 0.5-1.0 | | 0.25-4.0 | | 0.5-1.0 | | 0.25-1.0 | | 0.5-1.0 | | 1.0-10.0 | |
| AUC (tf) | 1820 | 35 | 4490 | 18 | 9320 | 22 | 23300 | 21 | 49000 | 29 | 83700 | 16 |
| (ng · hr/mL) | | | | | | | | | | | | |
| AUC(I) | 2040 | 34 | 4740 | 18 | 9890 | 24 | 24700 | 23 | 55400 | 36 | 96700 | 18 |
| (ng · hr/mL) | | | | | | | | | | | | |
| t½ (hr) | 21.4 | 18 | 21.7 | 23 | 26.5 | 25 | 27.1 | 22 | 35.4 | 29 | 39.4 | 23 |
| CL | 13.3 | 29 | 10.9 | 21 | 10.9 | 37 | 8.57 | 27 | 7.84 | 27 | 6.36 | 16 |
| (L/hr) | | | | | | | | | | | | |
| Vdss (L) | 396 | 20 | 331 | 12 | 389 | 13 | 324 | 18 | 378 | 22 | 356 | 23 |

^{a}median

TABLE 20
Mean Projected Steady-state Plasma and Blood Posaconazole PK
Parameters in Subjects Receiving a Daily Dose of POS IV
infusion over 1-1.25 hr

| | Plasma | | | | Blood | | | |
|---|---|---|---|---|---|---|---|---|
| | Cₘₐₓ | | | | Cₘₐₓ | | | |
| | (ng/mL) | | AUC (0-24 hr) | | (ng/mL) | | AUC (0-24 hr) | |
| Dose | | CV, | (ng · hr/mL) | | | CV, | (ng · hr/mL) | |
| (mg) | Mean | % | Mean | CV, % | Mean | % | Mean | CV, % |
| 25 | 155 | 26 | 1960 | 36 | 281 | 18 | 1580 | 29 |
| 50 | 335 | 16 | 4690 | 19 | 628 | 10 | 3560 | 17 |
| 100 | 714 | 23 | 9840 | 24 | 1360 | 21 | 8570 | 22 |
| 200 | 1670 | 22 | 24600 | 23 | 2760 | 9 | 20600 | 21 |
| 400 | 3540 | 27 | 54500 | 35 | 5870 | 12 | 50000 | 28 |
| 600 | 5100 | 16 | 94900 | 17 | 7260 | 10 | 85800 | 16 |

TABLE 21
Ratio of Mean Blood and Plasma Posaconazole PK
Parameters in Subjects receiving a Single Dose of POS IV
infusion over 1-1.25 hr

| | Blood/Plasma Ratio | |
|---|---|---|
| Dose | Cₘₐₓ | AUC (I) |

| (mg) | Mean | CV, % | Mean | CV, % |
|---|---|---|---|---|
| 25 | 2.42 | 17 | 0.839 | 7 |
| 50 | 2.76 | 25 | 0.772 | 13 |
| 100 | 2.71 | 13 | 0.878 | 6 |
| 200 | 2.50 | 18 | 0.844 | 6 |
| 400 | 2.50 | 19 | 0.933 | 8 |
| 600 | 2.45 | 17 | 0.904 | 5 |

TABLE 22
Ratio of Mean Projected Steady-state Blood and
Plasma Posaconazole PK Parameters in Subjects receiving
a Daily Dose of POS IV infusion over 1-1.25 hr

| | Blood/Plasma Ratio | |
|---|---|---|
| Dose | Cₘₐₓ | AUC (τ) |

| (mg) | Mean | CV, % | Mean | CV, % |
|---|---|---|---|---|
| 25 | 1.85 | 12 | 0.822 | 9 |
| 50 | 1.90 | 15 | 0.766 | 13 |
| 100 | 1.93 | 12 | 0.877 | 6 |
| 200 | 1.70 | 13 | 0.843 | 7 |
| 400 | 1.72 | 19 | 0.932 | 8 |
| 600 | 1.44 | 10 | 0.908 | 5 |

### Rising Multiple Dose Study

A rising, multiple dose assessment of the safety, tolerability, and pharmacokinetics of intravenous Posaconazole in healthy subjects was also conducted. The pharmacokinetic objectives of the study were two-fold: first, to determine the single-dose and multiple-dose pharmacokinetic (PK) profiles of POS IV in healthy subjects; and second, to determine the relative bioavailability of POS IV vs POS oral suspension administered under fed conditions. The formulation used in this study is that of Example 8.

A Phase 1, single-site, randomized, evaluator-blinded (within dose level), placebo-controlled, rising multiple-dose study evaluating the safety, tolerability, and pharmacokinetics of POS IV in three dose groups (100 mg, 200 mg, and 400 mg) of 10 healthy subjects per group was conducted in conformance with good clinical practices. Within each dose group, subjects were randomized on Day 1 to receive either POS IV or placebo (4:1) as a single dose on Day 1, followed by once daily doses on Day 5 through Day 14 according to a computer-generated schedule. Group 1 (100-mg dose group) subjects received a single oral dose of 100 mg POS oral suspension at least 7 days after the last IV dose of POS IV or placebo for assessment of the bioavailability of POS oral suspension. Plasma obtained from blood samples was analyzed for POS using a validated LC-MS/MS method. Plasma pharmacokinetic parameters were determined. Safety was assessed by ECGs, clinical laboratory tests, vital signs, and adverse event monitoring.

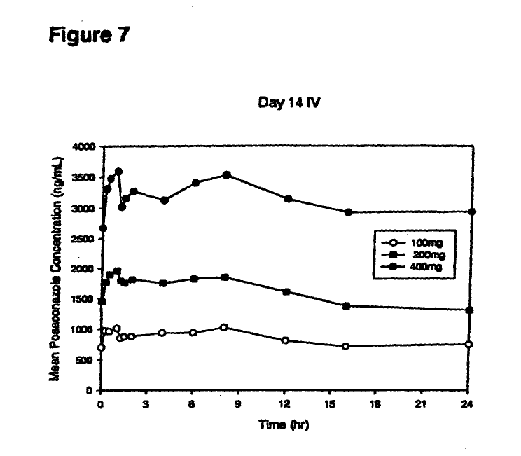

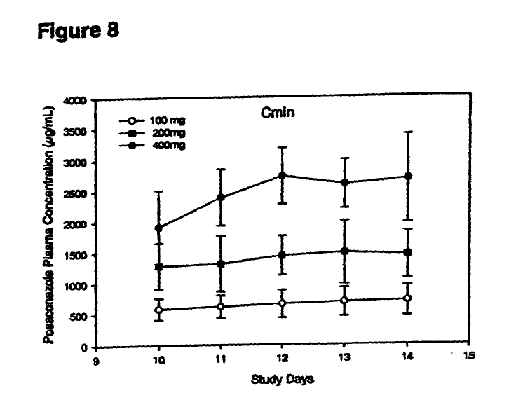

Mean POS plasma PK parameters obtained following a single dose and daily doses for 10 consecutive days are summarized in Tables 23 and 24, respectively. As in the rising single-dose study with POS IV, POS plasma concentrations in the rising multiple-dose study with POS IV declined rapidly after cessation of infusion and then increased subsequently, followed by a slow declining phase (see FIG. 7). The mean plasma Cₘₐₓ and AUC increased in an approximately dose-proportional manner. The steady-state appeared to be achieved by daily dosing for 6 days (see FIG. 8). The mean steady-state volume of distribution was large (334 to 348 L), suggesting extensive extravascular distribution and penetration into body tissues.

The pharmacokinetic modeling and simulation predicts a mean Cₘₐₓ of 2820 and mean AUC(0-24 hr) of 53,100 at the steady state when 300 mg posaconazole IV is administered as a 1-hour infusion to humans.

TABLE 23
Mean (n = 8) POS Pharmacokinetic Parameters after Single-Dose IV Infusion of POS IV to
Healthy Subjects

| | Mean (CV %) | | | | | | | | |
|---|---|---|---|---|---|---|---|---|---|
| | Cₘₐₓ | Tₘₐₓ^{a} | AUC (0-24) | AUC (tf) | AUC (I) | t_{1/2} | CL | V_{dss}^{b} | |
| Dose | (ng/mL) | (range) (hr) | (ng · hr/mL) | (ng · hr/mL) | (ng · hr/mL) | (hr) | (L/hr) | (L) | F |
| 100 mg IV | 443 | 1 | 4980 | 10100 | 11200 | 26.4 | 10.0 | 366 | — |
| (n = 8) | (15) | (0.5-1.0) | (22) | (35) | (40) | (19) | (35) | (20) | |
| 200 mg IV | 945 | 1 | 9290 | 20000 | 21900 | 26.0 | 9.91 | 359 | — |
| (n = 8) | (32) | (0.5-1.0) | (24) | (27) | (29) | (18) | (33) | (25) | |
| 400 mg IV | 1500 | 1 | 18400 | 43300 | 49500 | 30.5 | 8.2 | 353 | — |
| (n = 8) | (18) | (1.0-1.0) | (12) | (9) | (13) | (28) | (11) | (22) | |
| 100 mg PO | 268 | 5 | 4070 | 9020 | 10400 | 30.0 | 11.5 | 444 | 110^{c} |
| (n = 8) | (32) | (4.0-6.0) | (29) | (40) | (48) | (27) | (41) | (24) | (31) |

Abbreviations:
AUC (0-24) = area under the plasma concentration-time curve from 0 to 24 hours after dosing;
AUC (I) = area under the plasma concentration versus time curve to infinity;
AUC (tf) = area under the plasma concentration versus time curve to the final measurable sampling time;
CL = total body clearance based on plasma concentration;
Cₘₐₓ = maximum observed plasma concentration;
CV = coefficient of variation, expressed as a percent (%);
t_{1/2} = terminal phase half-life based on plasma concentration;
Tₘₐₓ = time of maximum observed plasma concentration;
V_{dss} = apparent volume of distribution at steady-state based on plasma concentration;
F = Relative bioavailability of POS IV compared to POS oral based on log-transformed AUC.
^{a}Median.
^{b}V_{dSS} for IV formulations and V_{d}/F for 100-mg oral formulation.
^{c}Based on log-transformed data.

TABLE 24
Mean (n = 8) POS Pharmacokinetic Parameters on Study Day 14 after
Multiple Daily-Dose IV Infusion of POS IV to Healthy Subjects

| | Mean (CV %) | | | | | |
|---|---|---|---|---|---|---|
| | Cₘₐₓ | Tₘₐₓ^{a} (range) | AUC (0-24) | t_{1/2}^{b} | CL | V_{dss}^{b} |
| Dose | (ng/mL) | (hr) | (ng · hr/mL) | (hr) | (L/hr) | (L) |
| 100 mg (n = 8) | 1080 (27) | 1 (0.25-8) | 20100 (30) | 36.8 (26) | 5.40 (31) | 334 (24) |
| 200 mg (n = 8) | 2030 (21) | 1 (0.5-8) | 38100 (28) | 38.6 (16) | 5.63 (29) | 339 (10) |
| 400 mg (n = 8) | 3830 (15) | 1 (0.5-8) | 75400 (15) | 33.3 (22) | 5.40 (14) | 348 (17) |

Abbreviations:
AUC (0-24) = area under the plasma concentration-time curve from 0 to 24 hours after dosing;
CL = total body clearance based on plasma concentration;
Cₘₐₓ = maximum observed plasma concentration;
CV = coefficient of variation, expressed as a percent (%);
t_{1/2} = terminal phase half-life based on plasma concentration;
Tₘₐₓ = time of maximum observed plasma concentration;
V_{dss} = apparent volume of distribution at steady-state based on plasma concentration.
^{a}Median.
^{b}t_{1/2} and V_{dss}, n = 6 for 100 mg and 200 mg and n = 4 for 400 mg. since terminal-phase slope could not be reliably estimated.

POS was slowly eliminated from plasma with a mean steady-state terminal half-life of 33 to 37 hours. The mean systemic clearance at steady-state ranged from 5.4 to 5.6 L/hr and was lower compared to that from the single-dose administration on Day 1 (range 8.2 to 11.5 L/hr; see Table 23), suggesting time-dependent pharmacokinetics. Consistent with a decrease in POS clearance on Day 14, steady-state exposure to POS was greater than that predicted from the single-dose data. This pharmacokinetic behavior is desirable as it allows the use of a lower maintenance dose in combination with a higher loading dose.

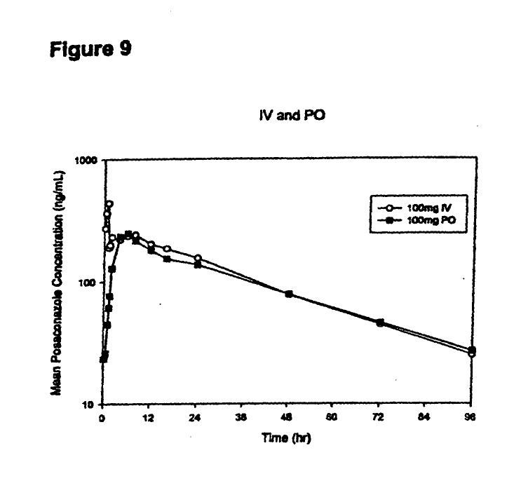

The relative bioavailability of POS IV compared to the current clinical POS oral suspension formulation was 110% at the 100-mg dose. The plasma concentration-time profile of POS from POS IV and POS oral formulations is shown in FIG. 9.

POS exposure from POS IV (AUC and Cₘₐₓ) exhibited low-to-moderate intersubject variability, with CVs for Cₘₐₓ and AUC values ranging from 15% to 40% (see Table 23). The variabilities in exposure (AUC and Cₘₐₓ) were higher for POS oral (% CV of up to 50%) compared to that for POS IV. POS was safe and well tolerated in this study.

These data from the rising multiple-dose study support the following conclusions:

Exposure to POS is approximately dose proportional in the 100- to 400-mg range. Steady-state appeared to be achieved by daily dosing for 6 to 7 days.

POS has a large volume of distribution, suggesting extensive distribution and penetration into body tissues.

Steady-state half-life of POS ranges from 33 to 37 hours and is higher compared to the single-dose half-life (26 to 29 hours). The mean systemic clearance at steady-state ranges from 5.4 to 5.6 L/hr and is lower compared to that from the single-dose administration on Day 1 (range 8.2 to 11.5 L/hr), suggesting time-dependent pharmacokinetics. The bioavailability of 100 mg POS IV is enhanced (i.e., 10% greater) relative to 100 mg POS oral suspension formulation.

Based on the foregoing pharmacokinetic data, certain dosing regimes are preferred for intravenous Posaconazole. Dosing regimes may include a loading dose and a set of maintenance doses. The loading dose can be either bolus or via infusion, although all foregoing data herein presented are based on infusion loading doses. The loading dose will be in the range of about 25 to about 600 mg. per day, preferably from about 200 to about 400 mg. per day. The maintenance doses will be in the range of about 25 to about 600 mg. per day, preferably from about 100 to about 300 mg. per day.

The above pharmacokinetic data, and pharmacokinetic modeling and simulations suggest that a variety of dosing regimes may be preferable, for example:
1. A single loading dose of 400 mg POS (IV infusion) QD followed by a maintenance dose of 100 mg POS (IV infusion) QD for 14 days;
2. A single loading dose of 400 mg POS (IV infusion) QD followed by a maintenance dose of 200 mg POS (IV infusion) QD for 14 days; and,
3. A single loading dose of 400 mg POS (IV infusion) QD followed by a maintenance dose of 300 mg POS (IV infusion) QD for 14 days.

The loading dose and maintenance dose of POS IV can be in the range of 25 to 600 mg once a day (QD). Alternative dosing frequencies are also possible, such as two times daily (BID), three times daily (TID) or every other day.

After an initial period of intravenous administration, patients can be switched to a maintenance therapy of oral suspension. For example, oral suspension can be dosed at 400 BID or up to 800 mg/day as a single or divided doses.

The maintenance dosing regime will be dependent upon such factors as the condition being treated and the relative health of the patient. For example, a maintenance dose of 200 mg. posaconazole 3 times per day may be appropriate for prophylaxis of invasing fungal infections. A maintenance dose of 200 mg. posaconazole once per day may be appropriate for oropharyngeal candidiasis. A maintenance dose of 400 mg. posaconazole twice per day may be appropriate for refractory oropharyngeal candidiasis.

### Particle Size PK Study

In order to determine the pharmacokinetic effects of Posaconazole particle size in intravenous formulations, a study was conducted by administering a single IV dose of formulations of varying PSDs to monkeys. More specifically, the characteristic attribute of each formulation was based either on the percentage of particles less than 1 micron or the median value of the distribution.

Five (5) stable posaconazole intravenous suspension formulations according to the present invention were dosed based on the median particle size (i.e., A, B, C of Table 25) or the percentage of particles less than 1 micron (i.e., D, E of Table 25). These formulations were variations of the formulation of Example 7, wherein the Posaconazole concentration ranged from 1.3 to 50 mg/mL. The pH was not adjusted. Table 25 below summarizes characteristics of the particle size distributions of the five formulations as measured after sterilization, but before dilution in 5% dextrose.

TABLE 25
Formulations used in a single-dose
pharmacokinetic study in male cynomolgus
monkeys (n = 6) receiving 8 mg/kg POS IV

| | | Volume |
|---|---|---|
| | | Percent Less |
| | Median Particle | than 1 |
| Treatment | Size (micron) | Micron |
| Treatment A | 2.8 | 9 |
| Treatment B | 2.3 | 13 |
| Treatment C | 1.7 | 20 |
| Treatment D | NA | 100 |
| Treatment E | NA | 50* |

*Based on theoretical calculation.

Each of three batches of formulation according to the present invention with differing median particle size (Treatments A, B, and C) were produced by controlling the parameters of high-pressure homogenization (e.g., pressure, duration or number of passes, and flow rate). Subsequently, filters of different removal ratings were implemented to further control particle size distribution of the final product after autoclaving. Comparative Formulation D (i.e., 100%<1 micron) was produced by centrifugation, hence separating the vast majority of suspended drug particles but maintaining primarily liposomal formations and particles less than 1 micron. Comparative Formulation E (i.e., 50% less than 1 micron) was produced as the composite of Formulations D and X (data not shown), mixed at a ratio of 86:5 by volume as predicted by theoretical calculations. Median particle sizes are not reported for Formulations D and E because they contain significant proportions of very small particles less than 1 micron that are outside the detection range of the Sympatec Analyzer lens.

The Formulations A, B, C and E (not D) were dosed to animals at a target post-dilution concentration of 4 mg/mL. The dilution medium was a solution of 5% dextrose. The maximum dose volume was set at 2 mL/Kg. For Formulation D, the potency was about 1.3 mg/mL; therefore, it was administered as received without further dilution so as not to exceed the target maximum dose of 2 mL/Kg. The IV bolus dose was administered through an in-dwelling catheter in WFI. The five formulations as reflected in Table 25 were dosed in monkeys at 8 mg/kg.

Individual plasma concentration-time data were used to calculate pharmacokinetic parameters using model-independent methods, as described in Gibraldi, M., Perrier, D., Pharmacokinetics, 2^{nd} ed. 409-17 (1982). The following pharmacokinetic parameters were determined: maximum observed plasma concentration (Cₘₐₓ); terminal phase half-life (t_{1/2}), time of maximum observed plasma concentration (Tₘₐₓ]; the AUC from time zero to infinity [AUC(I)]; systemic clearance (CL); and volume of distribution at steady-state (V_{dss}). The AUC (tf) and AUC (I) were calculated using the linear trapezoidal method. The AUC (I) was calculated from the predicted value at the time of final quantifiable concentration. The pharmacokinetic analysis was conducted using Pharsight Knowledgebase Server (PKS) with WinNonlin Enterprise Version 4.0.1 (Pharsight Corporation, Cary, N.C.).

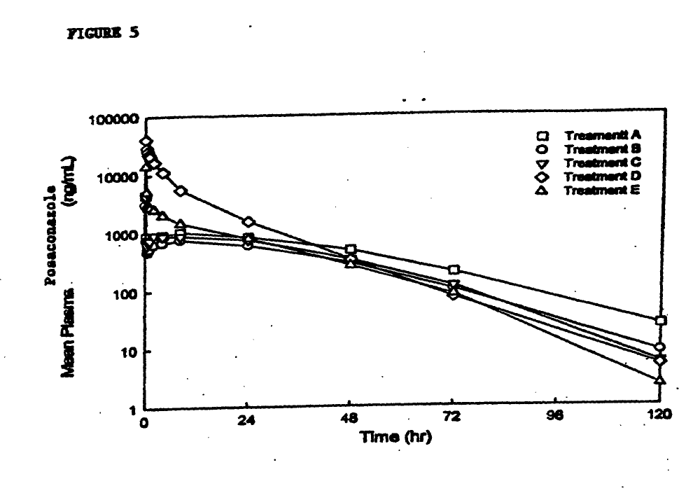

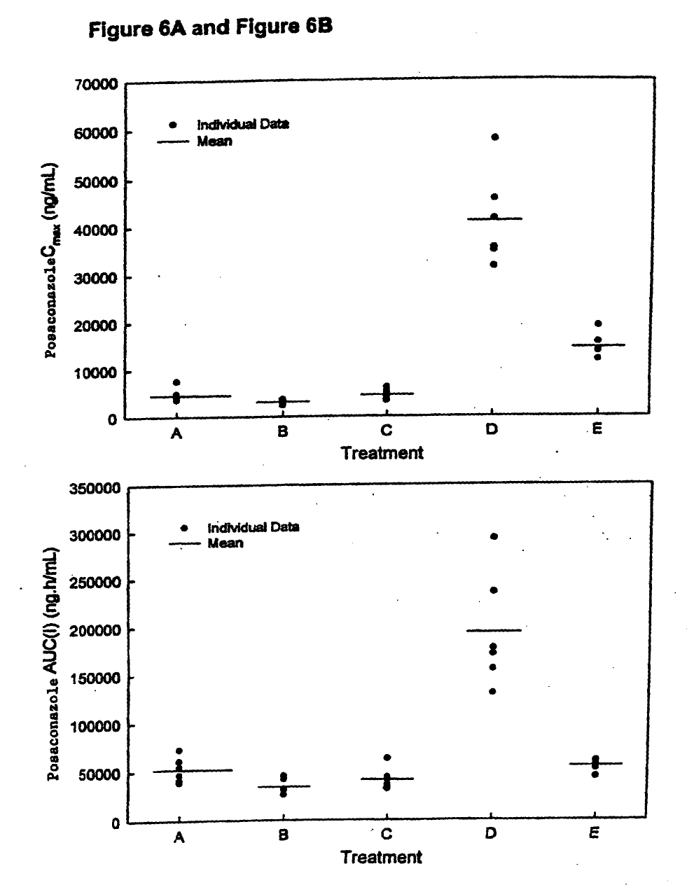

Summary pharmacokinetic data resulting from the administration of Treatments A-C are presented in Table 26. The corresponding data with respect to Treatments C-E are presented in Table 27. Mean plasma concentrations of Posaconazole are summarized in Table 28. The mean plasma concentration profiles of Posaconazole are illustrated in FIG. 5. The Posaconazole Cₘₐₓ and AUC(I) are illustrated in FIGS. 6A and 6B.

TABLE 26
Summary of Pharmacokinetic Parameters for Posaconazole in Male Monkeys following a Single Intravenous
Dose (8 mg/kg) of Posaconazole for Treatments A-C

| | Treatment A | | | | Treatment B | | | |
|---|---|---|---|---|---|---|---|---|
| Parameter | Mean | Min | Max | CV % | Mean | Min | Max | CV % |
| Tmax | 0.033 | .033 | .033 | 0 | 0.0330 | .033 | .033 | 0 |
| (hr) | | | | | | | | |
| Cmax | 4550 | 3530 | 7530 | 34 | 3100 | 2280 | 24,600 | 16 |
| (ng/mL) | | | | | | | | |
| AUC (I) | 51900 | 38,600 | 72,000 | 25 | 34200 | 25,700 | 45,000 | 22 |
| (ng * hr/mL) | | | | | | | | |
| t½ | 16.5 | 13.9 | 20.3 | 16 | 15.4 | 13.9 | 17.1 | 7 |
| (hr) | | | | | | | | |
| CL | 162 | 111 | 207 | 23 | 243 | 178 | 312 | 20 |
| (mL/hr/kg) | | | | | | | | |
| Vdss | 5250 | 3550 | 6140 | 19 | 6830 | 6010 | 7740 | 10 |
| (mL/kg) | | | | | | | | |

| | Treatment C | | | |
|---|---|---|---|---|
| Parameter | Mean | Min | Max | CV % |
| Tmax | 0.0330 | .033 | .033 | 0 |
| (hr) | | | | |
| Cmax | 4450 | 3190 | 6020 | 25 |
| (ng/mL) | | | | |
| AUC (I) | 41300 | 31,800 | 62,400 | 27 |
| (ng * hr/mL) | | | | |
| t½ | 15.2 | 14.4 | 16.1 | 4 |
| (hr) | | | | |
| CL | 204 | 128 | 251 | 22 |
| (mL/hr/kg) | | | | |
| Vdss | 5520 | 4250 | 6240 | 13 |
| (mL/kg) | | | | |

TABLE 27
Summary of Pharmacokinetic Parameters for Posaconazole in Male Monkeys following a Single Intravenous
Dose (8 mg/kg) of Posaconazole for Treatments C-E

| | Treatment C | | | | Treatment D | | | |
|---|---|---|---|---|---|---|---|---|
| Parameter | Mean | Min | Max | CV % | Mean | Min | Max | CV % |
| Tmax | 0.0330 | .033 | .033 | 0 | .033 | .033 | .033 | 0 |
| (hr) | | | | | | | | |
| Cmax | 4450 | 3190 | 6020 | 25 | 41,000 | 31,400 | 57,900 | 24 |
| (ng/mL) | | | | | | | | |
| AUC (I) | 41300 | 31,800 | 62,400 | 27 | 194,000 | 130,000 | 292,000 | 31 |
| (ng * hr/mL) | | | | | | | | |
| t½ | 15.2 | 14.4 | 16.1 | 4 | 11.3 | 9.23 | 15.2 | 22 |
| (hr) | | | | | | | | |
| CL | 204 | 128 | 251 | 22 | 44.4 | 27.4 | 61.5 | 28 |
| (mL/hr/kg) | | | | | | | | |
| Vdss | 5520 | 4250 | 6240 | 13 | 412 | 344 | 514 | 15 |
| (mL/kg) | | | | | | | | |

| | Treatment E | | | |
|---|---|---|---|---|
| Parameter | Mean | Min | Max | CV % |
| Tmax | .033 | .033 | .033 | 0 |
| (hr) | | | | |
| Cmax | 14,400 | 11,700 | 18,800 | 17 |
| (ng/mL) | | | | |
| AUC (I) | 55,600 | 43,900 | 60,400 | 12 |
| (ng * hr/mL) | | | | |
| t½ | 14.7 | 11.3 | 17.2 | 16 |
| (hr) | | | | |
| CL | 146 | 132 | 182 | 13 |
| (mL/hr/kg) | | | | |
| Vdss | 2730 | 2470 | 2980 | 9 |
| (mL/kg) | | | | |

TABLE 28
Mean Plasma Concentrations (ng/mL) for Posaconazole in Male Monkeys Following Single
Intravenous Dose (8 mg/kg) of Posaconazole with Varying Particle Size

| Time | Treatment A | | Treatment B | | Treatment C | | Treatment D | | Treatment E | |
|---|---|---|---|---|---|---|---|---|---|---|
| (hr) | Mean | CV % | Mean | CV % | Mean | CV % | Mean | CV % | Mean | CV % |
| 0.0330 | 4550 | 34 | 3100 | 16 | 4450 | 25 | 41000 | 24 | 14400 | 17 |
| 0.250 | 873 | 20 | 735 | 46 | 724 | 37 | 29700 | 12 | 5190 | 55 |
| 0.500 | 548 | 33 | 465 | 27 | 632 | 21 | 25800 | 16 | 3030 | 14 |
| 0.750 | 640 | 24 | 502 | 15 | 711 | 24 | 23000 | 17 | 2770 | 12 |
| 1.00 | 710 | 19 | 525 | 19 | 746 | 17 | 21200 | 14 | 2760 | 17 |
| 2.00 | 818 | 26 | 619 | 10 | 915 | 12 | 16700 | 20 | 2550 | 7 |
| 4.00 | 909 | 20 | 666 | 8 | 874 | 8 | 11300 | 26 | 2020 | 11 |
| 8.00 | 1020 | 28 | 743 | 10 | 876 | 12 | 5540 | 32 | 1450 | 6 |
| 24.0 | 818 | 20 | 584 | 15 | 731 | 25 | 1520 | 62 | 773 | 19 |
| 48.0 | 476 | 28 | 290 | 41 | 328 | 39 | 328 | 65 | 260 | 29 |
| 72.0 | 199 | 38 | 102 | 54 | 116 | 66 | 73.9 | 86 | 82.5 | 46 |
| 120 | 24.4 | 58 | 8.43 | 122 | 5.55 | 163 | 5.03 | 162 | 2.25 | 245 |

As shown in FIG. 5, the Posaconazole plasma concentration time profiles were characterized by a very rapidly declining distribution phase followed by a slowly rising re-distribution phase and a subsequent slow elimination phase when the percentage of particles less than 1 μm was less than 50%. The plasma concentration time profile for higher percentage (≧50%) of particles less than 1 micron was characterized by the absence of an initial rapid distribution phase

The mean Cₘₐₓ values were generally similar among Treatments A, B and C. The mean AUC(I)s were also similar among Treatments A, B, and C, thereby indicating that the exposures following these three treatments were similar. Following Treatment E, the mean Cₘₐₓ was approximately 3 to 5 times the mean Cₘₐₓ values resulting from Treatments A, B, and C, although there was no apparent difference in AUC(I). Following Treatment D, the mean Cₘₐₓ was approximately 9 to 13 times the mean Cₘₐₓ resulting from Treatments A, B, and C, whereas the mean AUC(I) was only about 4 to 6 times the mean AUC[I] resulting from Treatments A, B, and C. From a safety and tolerance perspective, a lower Cₘₐₓ and more sustained plasma levels are desirable, as antifungal efficacy is postulated to be associated with AUC/MIC ratio. These data suggest that Treatments A, B, and C, where the percentage of particles less than 1 micron is less than or equal to 20%, provide more sustained plasma levels at a lower Cₘₐₓ compared to that from Treatments D and E (about 100% and 50% less than 1 micron, respectively), and therefore are preferred from a safety, tolerance, and efficacy perspective.

Based on the above pharmacokinetic characteristics, it is evident that the particle size distributions embodied in Formulations A, B, and C are preferred. Thus, it can be generalized that particle size distributions will have preferred ranges of about 5 to about 25 vol % of particles of 1 micron or lesser size. Another preferred range spans from about 25 to about 50 vol % of particles of 1 micron or lesser size. Similarly, it may be concluded that particle size distributions will preferably have median particle sizes of about 1.5 to about 3.0 microns, more preferably 1.7 to about 2.8 microns, and specific preferred median particle sizes of about 1.7, 2.3, or 2.8 microns.

The mean t_{1/2} was similar for all treatments and ranged from 11.3 to 16.5 hours. The mean CL value was the lowest following Treatment D (44.4 mL/h/kg) and ranged from 146 to 243 mL/h/kg following Treatments A, B, C, and E. Similarly, the mean Vdss was lowest with Treatment D (412 mL/kg) and ranged from 2730 to 6830 mL/kg for Treatments A, B, C and E.

The data from the single intravenous dose of formulations of varying Posaconazole particle size distributions in male cynomolgus monkeys support the following observations.

First, it is evident from the data that those particle size distributions having a greater percentage of particles less than 1 μm result in higher Cₘₐₓ 's. This is consistent with the rapid uptake of larger particles by the reticulo-endothelial system (RES) which acts as an additional clearance mechanism clearing drug from blood, and ultimately results in a lower Cₘₐₓ for formulations with particle size distributions having a smaller percentage of particles less than 1 μm.

Second, based on mean AUC(I) values, the relative bioavailabilities of Treatments A, B, D, and E as compared to Treatment C were 126%, 83%, 470% and 135%, respectively.

Third, the relative bioavailabilities of Posaconazole IV formulation with median particle sizes of 3 and 2.5 μm compared to that of the formulation with a median particle size of 2 μm were 126% and 83%, respectively, indicating no discernable trend between the median particle size and plasma exposure.

Finally, the relative bioavailabilities of Posaconazole IV formulation with 100% of particles less than 1 μm and with 50% of particles less than 1 μm relative to that of the formulation with 25% of particles less than 1 μm were 470% and 135%, respectively, indicating that plasma exposure significantly increased as the percentage of particles less than 1 μm increased beyond 50%.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. It is to be understood that all formulations that are bioequivalent to those disclosed herein are also within the scope of the present invention.

Various publications are cited herein, the disclosures of which are incorporated by reference in their entireties.

What is claimed:

1-79. (canceled)

80. A formulation comprising a suspension of posaconazole particles, stabilized by a phospholipid, in a mixture comprising water, a thermoprotectant, and a buffer system, wherein said posaconazole has a particle size distribution whose particle size median value is between about 1.5 and about 3.0 microns and wherein said formulation maintains a particle size median value between about 1.5 and about 3.0 microns after being terminally sterilized by autoclaving at 121° C. for up to 150 minutes

81. The formulation of claim 80 wherein said particle size median value is between about 1.5 and about 3.0 microns after being subjected to one 20-minute autoclave cycle at 121° C. and up to five additional 30-minute autoclave cycles at 121° C., for a cumulative exposure at 121° C. of up to 170 minutes.

82. The formulation of claim 80, further comprising a second active ingredient selected from the group consisting of antifungals, antibacterials, antivirals, steroids, nonsteroidal anti-inflammatory drugs (“NSAIDs”), chemotherapeutics, and anti-emitics.

83. (canceled)

84. The formulation of claim 80, which provides at least one of a mean maximum plasma concentration (Cₘₐₓ) of Posaconazole of at least about 1080 ng/ml at steady state, and a mean plasma Area Under the Curve over 24 hours (AUC) value of Posaconazole of at least about 20,100 ng·hr/ml at steady state, after said formulation is infused over about 1 hour to deliver 100 mg of Posaconazole, and said infusion is repeated at an interval of once per day; or provides: at least one of a mean maximum plasma concentration (Cₘₐₓ) of Posaconazole of at least about 2030 ng/ml at steady state, and a mean plasma Area Under the Curve over 24 hours (AUC) value of Posaconazole of at least about 38,100 ng·hr/ml at steady state, after said formulation is infused over about 1 hour to deliver 200 mg of Posaconazole, and said infusion is repeated at an interval of once per day; or provides at least one of a mean maximum plasma concentration (Cₘₐₓ) of Posaconazole of at least about 2820 ng/ml at steady state, and a mean plasma Area Under the Curve over 24 hours (AUC) value of Posaconazole of at least about 53,100 ng·hr/ml at steady state, after said formulation is infused over about 1 hour to deliver 300 mg of Posaconazole, and said infusion is repeated at an interval of once per day; or provides at least one of a mean maximum plasma concentration (Cₘₐₓ) of Posaconazole of at least about 3830 ng/ml at steady state, and a mean plasma Area Under the Curve over 24 hours (AUC) value of Posaconazole of at least about 75,400 ng·hr/ml at steady state, after said formulation is infused over about 1 hour to deliver 400 mg of Posaconazole, and said infusion is repeated at an interval of once per day.

85. (canceled)

86. (canceled)

87. (canceled)

88. The formulation of claim 80, further characterized by providing at least one of a mean plasma half-life of about 36.8 hours and a mean plasma steady state volume of distribution of about 334 L, after said formulation is infused over about 1 hour to deliver 100 mg of Posaconazole, and said infusion is repeated at an interval of once per day.

89. The formulation of claim 80, further characterized by providing at least one of a mean plasma half-life of about 38.6 hours and a mean plasma steady state volume of distribution of about 339 L, after said formulation is infused over about 1 hour to deliver 200 mg of Posaconazole, and said infusion is repeated at an interval of once per day.

90. The formulation of claim 80, further characterized by providing at least one of a mean plasma half-life of about 33.3 hours and a mean plasma steady state volume of distribution of about 348 L, after said formulation is infused over about 1 hour to deliver 400 mg of Posaconazole, and said infusion is repeated at an interval of once per day.

91. The formulation of claim 80, which provides a mean Posaconazole steady state plasma concentration profile equivalent to that of either the 100 mg curve of FIG. 7 or the intravenous curve of FIG. 9, after said formulation is infused over about 1 hour to deliver 100 mg of Posaconazole and said infusion is repeated at an interval of once per day; or provides a mean Posaconazole steady state plasma concentration profile equivalent to that of the 200 mg curve of FIG. 7, after said formulation is infused over about 1 hour to deliver 200 mg of Posaconazole and said infusion is repeated at an interval of once per day; or provides a mean Posaconazole steady state plasma concentration profile equivalent to that of the 400 mg curve of FIG. 7, after said formulation is infused over about 1 hour to deliver 400 mg of Posaconazole and said infusion is repeated at an interval of once per day.

92. (canceled)

93. (canceled)

94. (canceled)

95. (canceled)

96. (canceled)

97. (canceled)

98. A formulation which is bioequivalent to the formulation of claim 84.

99. A formulation which is bioequivalent to the formulation of claim 85.

100. A formulation which is bioequivalent to the formulation of claim 86.

101. A method of treating or preventing an infection in an animal in need thereof by administering to said animal an effective amount of the formulation of claim 80.

102. The method of claim 101 wherein said infection is caused by a fungus or a parasite.

103. The method of claim 101 wherein said infection is one or more selected from the group consisting of: oropharyngeal or esophageal candidiasis; refractory oropharyngeal and esophageal candidiasis; invasive aspergillosis, candidiasis, fusariosis, scedosporiosis, infections due to dimorphic fungi, zygomycosis, and invasive infections due to rare molds or yeasts; invasive mycoses in patients who are refractory to, or intolerant of, other therapies; Candidiasis, invasive mold infections in patients who have undergone intensive chemotherapy and/or radiation therapy for hematologic malignancies, bone marrow or peripheral stem cell transplant conditioning regimens, and patients receiving combination immunosuppressive therapy for the treatment of acute or chronic graft-versus-host disease or prevention of solid organ transplantation; Chagas disease; and, Leishmaniasis.

104. The method of claim 101, wherein said formulation is administered intravenously.

105. The method of claim 101 wherein said formulation is administered intramuscularly, subcutaneously, ophthalmically, subconjuctivally, intraocularly, via anterior eye chamber injection, intravitreally, intraperitoneally, intrathecally, intracystically, intrapleurally, intranasally, topically, via wound irrigation, intradermally, intrabuccally, intra-abdominally, intra-articularly, intra-aurally, intrabronchially, intracapsularly, intrameningeally, intrapulmonarilly, via inhalation, via endotracheal or endobronchial installation, via direct installation into pulmonary cavities, intraspinally, intrasynovially, intrathoracically, via thoracostomy irrigation, vaginally, epidurally, rectally, intracistemally, intravascularly, intraventricularly, intraosseously, via irrigation of infected bone, and via application as part of any admixture with cement for prosthetic devices.

106. The method of claim 101, wherein said animal is a human.

107. (canceled)

108. The method of claim 101, wherein said formulation is administered by first administering an intravenous loading dose and then administering a maintenance dose, and optionally administering a second maintenance dose of from about 100 mg/day to about 800 mg/day as a single or divided dose, wherein the second maintenance dose is optionally a posaconazole suspension administered orally.

109. The method of claim 108, wherein said loading dose is from about 200 mg to about 400 mg. and said maintenance dose is an intravenous dose of from about 100 mg/day to about 400 mg/day.

110. (canceled)

111. The formulation of claim 80 wherein said phospholipid is 1-Palmitoyl-2-oleoyl-sn-glycerol-3-phosphocholine (POPC) and wherein the concentration of posaconazole is about 50 g/L, the concentration of 1-Palmitoyl-2-oleoyl-sn-glycerol-3-phosphocholine (POPC) is about 40 g/L, and the concentration of trehalose is about 250 g/L.
